(19) United States
(12) Patent Application Publication
(10) Pub. No.: US 2003/0143626 A1
(43) Pub. Date: Jul. 31, 2003

(54) Targeted modification of intracellular compounds

(76) Inventors: Pierre Colas, Saint Didier Mont D'Or (FR); Roger Brent, Berkeley, CA (US); Barak A. Cohen, Cambridge, MA (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/066,965

(22) Filed: Nov. 13, 2001

(30) Foreign Application Priority Data
Nov. 13, 2000 (EP) 00403156.3

(51) Int. Cl. A61K 48/00; A61K 38/17; A61K 38/08; G01N 33/53; C07H 21/04; C12P 21/02; C12N 5/06; C07K 14/47; C07K 7/08

(52) U.S. Cl. 435/7.1; 435/69.1; 435/320.1; 435/325; 514/12; 514/13; 514/14; 514/44; 536/23.5; 530/324; 530/325; 530/326; 530/327

(57) ABSTRACT

Disclosed are processes for specifically modulating the properties of a target molecule T, and/or of a component C, which interacts directly or indirectly with T. Also disclosed are processes for the production of a targeted effector having the capacity to specifically modulate the properties of an intracellular target molecule and/or a cellular component C which interacts directly or indirectly in the cell with the molecule. Also disclosed are effector molecules, nucleic acids encoding same and constructs e.g., vectors and host cells, containing the nucleic acids, as well as pharmaceutical compositions, and uses of the effector molecules. Further disclosed are intracellular recognition molecules containing proteinaceous recognition domains, as well as processes for identifying dimeric recognition moieties having the capacity to force an interaction between two target molecules T1 and T2, and/or a cellular component C which interacts directly or indirectly in a cell with T1 and T2.

The present invention relates to methods for the controlled modulation of cellular physiology using targeted modification of intracellular components. The invention also concerns molecules capable of bringing about such targeted modifications.

Specific targeting of intracellular molecules provides a powerful means of modifying cellular function and physiology. Attempts have been made to achieve this aim using molecules which have the capacity to bind a given target protein with high specificity within a cell. Such specific-binding molecules have been identified by screening large complex libraries, for example peptide aptamers (ref. 1), or arrays of synthetic compounds, for example those generated by combinatorial chemistry, or variants of binding domains of known proteins. A rational design approach can also be used, whereby knowledge of the structure of both the target and binding molecules is used to construct rational derivatives, such as variants of the PDZ domain (ref. 36). A further example is provided by intracellular antibodies or <<intrabodies>> comprising the variable domains of immunoglobulin heavy and light chains synthesised as a single polypeptide (ref. 41).

Binding of these intracellular <<recognition>> molecules to the target compound generally brings about a modification in the function of the target, for example as a result of the occlusion of an active site, the blocking or stabilization of interactions with other cellular components (for example protein-protein or protein-DNA interactions), or the fixation of the target in an active or inactive conformation.

Whilst a considerable array of different intracellular effects can be achieved using these targeting molecules, there is nevertheless a limit to the extent of the functional manipulations which can be carried out in this way. Indeed, the modulation is necessarily limited to one which can be achieved by binding of the recognition molecule to its target. Many desirable intracellular changes, such as covalent modification of an endogenous target, exposure of the target to a particular enzyme, destruction of the target or transport of the target to a given sub-cellular compartment, cannot be achieved using the existing recognition molecules alone.

A further limitation of the known systems is the binding affinity of the artificial protein recognition molecules to their target molecules. This affinity is often in the micromolar range, although can be higher for peptide aptamers. Whilst a binding affinity in this range is adequate for some cellular targets, many intracellular proteins bind their natural targets with a higher affinity. Competitive inhibition of such interactions by artificial recognition molecules requires binding affinities higher than those previously reported.

There is thus a need to extend the ability to control biological processes by intracellular targeting molecules, both in terms of the type of modification which can be achieved and the strength of the interaction with the target molecule.

The present inventors have developed a system which overcomes the limitations of the known targeting molecules.

According to a first aspect of the present invention, an intracellular recognition molecule which interacts with its target with an affinity of less than 1×10⁻⁶ M, and preferably less than 1×10⁻⁸ M, is covalently linked to an effector molecule, capable of exerting a desired effect. The recognition module directs the effector module to its specific intracellular target, where the combined recognition and effector modules can exert the desired effect in a specific manner and with an affinity sufficiently high to bring about the desired change to the appropriate extent.

The specific targeting molecules of the invention are thus composed of two functional entities, the first being the recognition moiety, which will be designated <<R>>, and the second being the effector moiety which will be designated <<E>>. The moieties <<R>> and <<E>> are covalently linked to each other, either directly or through linker elements, and are together designated a <<targeted effector>>.

According to the invention, the targeted effector is introduced into a cell, where the recognition moiety confers the necessary specificity and affinity to the effector moiety, thus allowing a specific change to be brought about on the target molecule T, or a cellular component, C, which interacts with the target T, either directly or via an associated complex.

The technique of the invention is particularly advantageous in that the affinity of the interaction between the targeted effector (i.e. the covalent association of the recognition moiety and the effector moiety), and the target has been found to be directly proportional to that of the recognition moiety before fusion to the effector moiety. This is particularly surprising in view of previous reports which suggested that the fusion of intracellular recognition molecules to other molecular entities would lead to a considerable lowering of binding affinity.

It has also been found in the context of the present invention that the degree of change brought about by the combined action of the recognition moiety and the effector moiety is proportional to the affinity of the recognition moiety for the target. Thus, in accordance with the invention, not only can the nature of the intracellular change be controlled by use of an appropriate effector moiety, but the extent of the change can also be regulated by choice of a recognition moiety having a suitable binding affinity for its target.

Surprisingly, the inventors have also established that the specificity of the recognition moiety for its target is not adversely affected by the fusion to the effector moiety. Even in cases where the effector moiety has an inherent specificity different from that of the recognition moiety, it has been observed that, by virtue of its covalent link to the recognition moiety, the effector moiety acquires the capacity to exert its effect on the target molecule T. The invention thus enables the intracellular specificity of an effector molecule to be redirected.

Generally speaking, the invention relates to a process for specifically modulating the properties of a target molecule T, and/or of a component C which interacts directly or indirectly with T, said process comprising:

contacting T with a chimeric molecule, a so-called <<targeted effector>>, comprising

a recognition moiety R having the capacity to specifically interact with a site on the target molecule T, R interacting with T with a first affinity A₁ and

an effector moiety, E, covalently linked to said recognition moiety R, E being a molecule, or a portion thereof, which has an initial capacity to exert an effect on at least one molecule M, and which when it is covalently linked to R, acquires the capacity to specifically exert the effect on the target molecule T,

wherein the targeted effector interacts with T with a second affinity A₂, the affinity A₁ or the affinity A₂ corresponding to a K_{d} of less than 1×10⁻⁸ M, and the properties of T and/or of C are specifically modulated by the effector moiety E.

The process of the invention is suitable for use in vitro and in vivo. For the modulation of the properties of an intracellular target T, and/or of a cellular component C which interacts directly or indirectly in a cell with T, the process of the invention comprises

introducing into a cell a chimeric molecule, a so-called <<targeted effector>>, comprising

a recognition moiety R having the capacity to specifically interact, within the cell, with a site on an intracellular target molecule T, R interacting with T with a first affinity A₁ and

an effector moiety, E, covalently linked to said recognition moiety R, E being a molecule, or a portion thereof, which has an initial capacity to exert an effect on at least one molecule M, and which when it is covalently linked to R, acquires the capacity to exert the effect on the intracellular target molecule T,

wherein the targeted effector interacts with T with a second affinity A₂, the affinity A₁ or the affinity A₂ corresponding to a K_{d} of less than 1×10⁻⁸ M, and the properties of T and/or of C are specifically modulated by the effector moiety E.

The present invention thus enables the controlled modulation of the properties of an intracellular target molecule T or a molecule C interacting with T in the cell.

The above generally-defined process is particularly advantageous when carried out using targeted effectors whose recognition moieties are composed of peptides conformationally constrained by covalent linkage to a heterologous platform

In the context of the invention, the following terms encompass the following meanings

<<modulation of the properties>> of a molecule T or C signifies the modification of one or more of the chemical, biochemical, physical and/or functional properties of T or C;

<<the chemical properties>> of a molecule T or C are properties arising from the chemical composition of the molecule. Modification of the chemical properties of T or C includes the formation or breakage of one or more covalent bonds or non-covalent bonds, the incorporation or loss or exchange of atoms, functional groups or residues;

<<the biochemical properties>> of a molecule T or C are properties arising from the chemistry of the molecule as part of a living organism, or cell or tissue, for example the capacity of T or C to combine with, or to act on, or to be acted upon, by other substances in the cell;

<<the physical properties>> of a molecule T or C are the properties which together define the physical or material condition of the molecule, its energy, its spatiotemporal properties, for example the sub-cellular localization of T or C in a cell, its exposure at the surface of the cell, its internalization, conformation, oligomerization state etc.;

<<the functional properties>> of a molecule T or C signifies any one or more of the functions of T or C, particularly as exhibited in a cell, for example at any given stage of development or in a given cellular compartment or in a given set of environmental conditions. The modificiation of the functional properties of T and/or C includes the enhancement or reduction in the efficiency of a function, or the abolition or creation of a function. An example of a function which can be modulated according to the invention is the ability of T or C to interact with cellular components such as proteins or nucleic acids, resulting in a significant change in the molecule's ability to act as an enzyme or co-factor, to regulate gene expression, to transport intracellular components, to synthesise cellular components etc . . . . The chemical, biochemical and physical properties of an intracellular target T or component C are usually directly related to the function of the molecule. Consequently, the modification of the chemical, biochemical and/or physical characteristics may give rise to a change in the function of that molecule.

A binding molecule is said to <<interact>> with a target molecule when the binding molecule binds non-covalently to the target. Protein-protein and protein-DNA interactions are typical of this type of binding. The interaction occurs between the binding domain of the binding molecule and a precise site, or <<epitope>> on the target molecule.

A binding molecule is said to interact <<specifically>> with a site on a target molecule when the binding molecule only has the capacity to interact with that site, i.e. the binding domain of the binding molecule cannot interact with any other site. The site (or <<epitope>>) in question may occur on a plurality of target molecules, in which case the binding molecule specifically recognises a range of target molecules, and can be considered to be a <<pan-interacting>> molecule. Alternatively the epitope may occur exclusively on one target molecule, and the binding molecule thus can only recognise that target. Occasionally, a binding molecule has the capacity to bind with high affinity to a given epitope, and also has the capacity to bind to a different epitope with lower affinity. In such a case, the interaction between the binding molecule and the target site is said to be <<specific>> when the binding affinity is at least in the micromolar range (i.e. a dissociation constant k_{d} equal to or less than 1×10⁻⁶ M, preferably equal to or less than 1×10⁻⁷ M and most preferably equal to or less than 1×10⁻⁸ M).

A peptide aptamer, unless otherwise indicated, signifies a recognition moiety comprising one or more peptide recognition regions <<V>>, conformationally constrained by covalent bonding to a platform P. The peptide V is also known as a variable region since, in a population of aptamers, this region can be varied.

According to the invention, the recognition moiety R interacts with the target T with an affinity A₁, and the targeted effector interacts with T with an affinity of A₂.

In accordance with the present invention, binding affinities <<A>> are expressed as K_{d}s (M), as measured in vitro at equilibrium. K_{d}s can be calculated by any conventional means, such as dividing dissociation rate constants (s⁻¹) by association rate constants (M⁻¹ s⁻¹). Thus, the higher the binding affinity A, the lower the corresponding K_{d}.

It is a preferred feature of the invention that either A₁ or A₂ or both A₁ and A₂, have a K value of less than 1×10⁻⁸ M. A₁ may be substantially equal to A₂ or may be less than A₂ Preferably, A₁ has a K_{d} value of less than 1×10⁻⁸ M, most preferably less than 5×10⁻⁹ M, for example less than 1×10⁻⁹ M. According to a preferred embodiment of the invention one or both of the binding affinities A₁ and A₂ correspond to K_{d} values comprised between 1×10⁻⁹ M and 1×10⁻¹⁴ M, for example between 1×10⁻⁹ M and 1×10⁻¹² M, or between 1×10⁻⁹M and 1×10⁻¹¹ M.

Binding affinities of recognition moieties and targeted effectors can be measured in vitro, using any appropriate means known in the art, for example by carrying out S.P.R. or evanescent wave experiments such as those described below in the Examples, and in reference (1). This type of method is generally used in accordance with the invention for the determination of the binding affinity A₁ i.e. the K_{d}s for a <<naked>> recognition moiety R, without the covalently fused effector moiety E. However, depending on the nature of the effector moiety E, the method can in some cases also be used to determine binding affinity A₂ i.e. K_{d}s for the targeted effector.

If the interaction of the recognition moiety R, or the targeted effector, with the target T gives rise to a detectable phenotype, the binding affinities A₁ and/or A₂ can also be evaluated using the extent of phenotypic interaction as an indication of the strength of the binding.

In so far as the K_{d} of the <<naked>> recognition moiety R correlates closely with that of the fused targeted effector, it is not indispensable to quantitatively determine both A₁ and A₂. As long as one of these K_{d} values is less than 1×10⁻⁸ M, the binding and modulation properties of the targeted effector will be suitable for carrying out the process of the invention.

According to a particular variant of the invention, the binding affinity of an interaction between R and T can be improved by modifying the chemical composition of the binding region of R, for example by mutagenesis using appropriate techniques such as mutagenic PCR etc, and screening the thus-obtained mutants for variants having the desired binding characteristics.

According to a first variant of this aspect of the invention, the recognition moiety R is mutated to give a mutant recognition moiety Rₘ, Rₘ having the capacity to specifically interact with a site on the intracellular target molecule T, with an affinity Aₘ, which is stronger than the affinity of the interaction between R and T (A₁). Expressed in terms of Kd, the Kd value corresponding to Aₘ is less than that corresponding to A₁.

Alternatively, according to a second variant of this aspect of the invention, the recognition moiety R may already be a mutant of a parent recognition moiety Rₚ, Rₚ specifically interacting with a site on the intracellular target molecule T, with an affinity Aₚ, which is not as strong as the affinity of the interaction between R and T (A₁). Expressed in terms of Kd, the Kd value of Aₚ is greater than that of A₁, i.e. greater than 1×10⁻⁸ M.

Using the technique of mutagenesis, the inventors have succeeded in preparing recognition moieties R, particularly peptide aptamers, which have binding affinities of less than 1×10⁻⁸ M, in particular less than or equal to about 5×10⁻⁹ M. This technique can be used to produce peptide aptamer recognition moieties having affinities as high as 1×10⁻¹² or 1×10^{-13.} Enhancement of the binding affinity of the parent molecule is generally from around ten- to around twenty- or twenty-five fold after mutagenesis and sorting.

As shown in the Examples, mutagenesis of the binding domain of R may leave the specificity characteristics of R unaltered. Alternatively, mutagenesis may affect the specificity of R, either enhancing or reducing it. For example, the binding domain of a first recognition moiety R may initially interact exclusively with a first target site S₁ on the target T. Upon mutagenesis of the binding domain, the mutated recognition moiety Rₘ may acquire the capacity to bind to a second binding site S₂, present on the same target molecule T, but also present on other molecules which previously were not targets for R.

The modification of the binding behaviour of the recognition moiety when optimisation of the binding affinity is carried out, can be detected easily by specificty screens. Depending upon the objective sought, the change in specificity can either be exploited advantageously, or can be eliminated by sorting the mutants to identify those conserving the initial specificity but having enhanced affinity.

In accordance with the invention, the recognition moiety R having enhanced affinity, is covalently bound to an effector moiety, providing a targeted effector of particularly high affinity for example less than 1×10⁻⁹ M.

The recognition moiety R is, generally speaking, any molecule which has the capacity to interact with high specificity and affinity within a cell with a target T. Preferably, the recognition moiety R is, or comprises, a protein or a peptide, and includes protein/non-protein composites. The recognition moiety may comprise the binding region of a protein, or a modified version thereof, or the antigen-binding domain of an antibody. Alternatively, R may be an entirely synthetic molecule generated at random or through rational design.

According to a particularly preferred embodiment, the recognition moiety R comprises or consists of a randomly generated peptide, or a part thereof. By randomly generated peptide is meant a peptide having a random sequence of amino acid residues. Such peptides are encoded by random DNA sequences such as can be generated by an oligosynthesizer. A random sequence has no significant bias in the composition of the sequence. Random peptides may or may not exhibit homology with known proteins or naturally occurring protein binding domains. Such peptides can be selected from combinatorial libraries, providing a huge pool of potential recognition molecules. Preferably, the random peptides have no significant homology with naturally occurring petides.

The recognition moiety R may also comprise or consist of a known peptidic interaction surface.

The recognition peptides R are generally relatively small in size, for example from 5 to 150 amino acids, preferably around 5 to 120 amino acids, most preferably from 5 to 60, and even more preferably 6 to 40 amino acids, although may in some instances be larger or smaller. Ideally the peptide recognition moieties are easily expressed and purified in bacterial cells and in mammalian cells.

Advantageously, the recognition moiety R comprises at least one recognition region, displayed in a conformationally constrained manner in a platform, <<P>>. The composition of the recognition region <<V>> can be varied to provide the appropriate recognition characteristics, whilst the platform <<P>> remains constant. The recognition region will be referred to herein as the <<variable region>> or <<V>>. Recognition moieties comprising one or more variable recognition regions <<V>>, conformationally constrained in a platform P will be designated <<aptamers>>. If, in such a recognition moiety, the one or more of the variable regions V comprises a peptide, the molecule will be referred to as a <<peptide aptamer>>.

The variable region <<V>> is conformationally constrained in the platform P, also known as a scaffold. This means that V has reduced flexibility i.e. can assume a limited number of conformations, because at least one of its extremities is covalently linked to the platform. Preferably, <<V>> is doubly constrained i.e. both extremities of the variable region are covalently linked to the platform. For example, for peptide aptamers, both the N-and the C-terminal extremities of <<V>> are covalently linked to the platform. Typically, the variable region is inserted in the central portion of the scaffold, or in an “active site loop”, permitting optimal interaction characteristics to be attained. Alternatively, or additionally, the extremities of the variable region may be conformationally constrained by covalently linking them together, for example by disulfide bridges.

The conformational constraint of R may or may not involve the formation of disulphide bridges.

The variable region <<V>> is preferably a peptide having from approximately 5 to approximately 60 amino acids, preferably 10 to 40 amino acids, for example 20 amino acids or thereabouts.

The platform P (also known as a <<scaffold>>) can be any molecule which is capable of reducing, through covalent bonding, the number of conformations which <<V>> can assume. Examples of conformation-constraining scaffolds include proteins and peptides, for example thioredoxin and thioredoxin-like proteins, nucleases (e.g. RNaseA), proteases (e.g. trypsin), protease inhibitors (e.g. eglin C), antibodies or structurally-rigid fragments thereof, fluorescent proteins such as GFP or YFP, and conotoxins. A conformation-constraining peptide can be of any appropriate length, for example from 5 to 150 amino acids, preferably 5 to 40 or 5 to 60 amino acids. Even shorter stretches of amino-acid residues can provide conformational constraint. Other suitable platform molecules include carbohydrates such as sepharose, magnetic beads, fluorescent beads or gold beads. The platform P may be a linear or circular molecule, for example, closed to form a loop.

The platform is generally heterologous with respect to the peptide V. This means for example that the platform is not of the same origin as the V peptide. The association of the platform and V generally does not exist in nature.

In addition to its role in conformation constraint, the platform may also be capable of conferring protection from proteolytic degradation and the like, of conferring desirable solubility characteristics etc.

Thioredoxin (TRX) and thioredoxin-like (TRX-like) proteins are particularly preferred as platforms of the present invention. Thioredoxin-like proteins are defined herein as proteins having at least 18%, preferably at least 40% and most preferably at least 75% homology with the amino acid sequence of E. coli thioredoxin over an amino acid sequence length of 80 amino acids (ref 43). Thioredoxin-like molecules also include peptides which have a three-dimensional structure substantially similar to that of human or E. coli thioredoxin, for example glutaredoxin (ref 44 ). A particularly preferred thioredoxin platform is human thioredoxin.

In TRX- or TRX-like recognition moieties, the variable region <<V>> is usually inserted at the active site loop of the TRX or TRX-like protein.

According to a preferred embodiment of the invention, peptide aptamers as described above are selected from combinatorial libraries by two-hybrid methods, using aptamer derivatives that also bear activation domains such as Act, and the desired target proteins fused to DNA-binding domains such as LexA as baits. Such selection ensures that the aptamers function in vivo. Peptide aptamers typically exhibit Kds for their target of about 1×10⁻⁷ to 1×10⁻⁸ M (ref. 1). These molecules can discriminate between closely related members of protein families (1), and even between different allelic forms of proteins (2, and Examples below). Peptide aptamers bind to a specific site on a target molecule and are therefore capable of inhibiting the interaction of a target with one interaction partner, but not all. The functional modulation of an intracellular target can therefore be carried out in an extremely precise manner.

Peptide aptamers have been shown to be capable of disrupting specific protein interactions in vivo (reviewed in ref. 10). For example Anti-Cdk2 aptamers competitively inhibit the interaction of Cdk2 with one of its substrates and, when expressed in human cells, delay progress through the cell cycle (3). Similarly, anti-E2F, anti-HPV E6, and anti-Ras aptamers disrupt the function of their protein targets in mammalian cells (4, 5) and anti-Drosophila Cdc2 and Cdc2c aptamers inhibit the function of their targets in imaginal disks (6). Finally, it has been shown that aptamers can be used as dominant genetic agents to cause a phenotype and to identify, in subsequent two hybrid assays, the proteins and interactions they target (7, 8, 9). These agents are thus well-suited for carrying out the controlled modulation of cellular physiology according to the invention.

According to the invention, the recognition moiety R, which is preferably a peptide aptamer, is covalently bound to an effector moiety, E to provide the targeted effector. The effector moiety E is any molecule, or part of a molecule, which has an initial capacity to exert an effect on at least one molecule M, thus affecting the chemical biochemical, physical or functional properties of M.

In accordance with the invention, E is said to have an <<initial>> capacity to exert an effect on M. In this context, an <<initial>> capacity signifies that E has this capacity prior to its being covalently linked to the recognition moiety R. The <<initial>> capacity of E to exert an effect on M may be an inherent capacity, or may be a capacity which is induced for example in response to an external stimulus such as heat or light etc. The <<initial>> capacity may also be a capacity which has been artificially conferred on E, for example by modification of the molecule E.

Generally speaking, E has an initial capacity to exert an effect in cis and/or in trans on M, and when it is covalently bound to R, E acquires the capacity to exert the same effect in trans on the target T. E may or may not retain its ability to act on M after fusion to R.

According to the invention, an effector moiety E, which has the initial capacity to exert an effect <<in trans>> on a molecule M is a molecule which, when it is not covalently bound to the recognition molecule R, has the ability to act on, or to be acted upon by, a molecule M which is not covalently bound to E, or to a molecule comprising E. Likewise, an effector moiety E which has the inherent capacity to exert an effect <<in cis>> on a molecule M, is a molecule which, when it is not covalently bound to the recognition molecule R, has the ability to act on, or to be acted upon by, itself or on a molecule M comprising E.

The molecule M upon which E initially has the capacity to exert an effect may be an intracellular or extracellular molecule. It may be the same or different from the molecule T which is the target of the recognition moiety R.

The inventors have established that in some cases, effector moieties which normally exert an effect in cis, i.e. on a molecule which is covalently bound to the effector, acquires the ability to exert that same effect in trans on the recognition moiety's target T. The cis effect is thus converted to one which can be exerted without covalent bond to the target molecule.

In the targeted effector, E is covalently linked to the recognition moiety R, for example either directly to the amino or carboxy terminal of R, or alternatively via linking sequences which may themselves also provide a particular function. E may also be inserted within R, provided that the function of the variable region V is not disrupted by the insertion. E may thus be conformationally constrained at one or both extremities.

E is preferably, but not necessarily, a protein or a peptide, or comprises a protein or a peptide. For example, the effector moiety E may comprise or consist of an enzyme, a catalytic domain thereof, a co-factor, an addressing signal, a transcription regulatory protein, a tracer protein, a molecule having therapeutic or diagnostic properties, a second recognition moiety, a second targeted effector, a radionuclide, a chemical modifier, an enzyme which can act upon itself etc. When E is or comprises a peptide, its size can vary from two to three amino acids to upto several hundred amino acid residues or more. Typical sizes range from about twenty to 200 amino acids, for example, 30 to 100 amino acids.

Examples of initially trans-acting effector moieties are enzymes, transcription factors or components thereof, substrates for enzymes, co-factors etc. Examples of initially cis-acting effector moieties are addressing signals or intracellular transporters, labels, markers etc.

According to a particularly preferred embodiment of the invention, the effector moiety E comprises a second recognition moiety R₂ having the capacity to specifically interact, within a cell, with a site on an intracellular target molecule T₂, T₂ being a target molecule which is distinct from T. The targeted effector is thus a dimeric molecule.

The inventors have established that upon linking the effector moiety E to the recognition moiety R, E acquires the specificity of R, that is, it acquires the capacity to act, in a cell, upon the same range of intracellular targets as R. This effect is particularly surprising when E is a molecule which has an intrinsic specificity different from that of R. For example, the inventors have observed that an enzyme which normally is incapable of interacting with a given intracellular target T acquires the ability to exert its usual enzymatic activity on T when it is joined to the recognition moiety. The specificity of the effector moiety can thus be redirected in accordance with the invention. In addition to acquiring the specificity of R, the covalently linked E may in some cases also conserve the ability to exert an effect on its initial partner M. Whether or not E retains the ability to act on M depends upon the nature of E, M, and R and the nature of the cell. Moreover, if the effector moiety E is a molecule which has an endogenous counterpart in the cell, the specificity of the endogenous counterpart is not affected by the presence in the cell of the targeted effector carrying E with a redirected specificity.

The targeted effector of the invention may carry one or more effector moieties, for example two or three. If more than one effector moiety is present, they can be functionally co-ordinated or functionally independent, provided that the functions are compatible with each other. An example of functionally co-ordinated effector moieties is provided by a first effector moiety E₁ which is responsible for directing the target T to a particular sub-cellular compartment, and a second effector moiety E₂ which can act on the target T, or a different molecule, only once it is in that cellular compartment. An example of this type of system is where E₁ is a nuclear localising signal, E₂ is a component of a modular transcription factor, and the target T is a cytoplasmic protein which comprises a second component of the modular transcription factor. The targeted effector interacts with the cytoplasmic target T, thereby reconstituting the modular transcription factor and simultaneously carrying the cytoplasmic target molecules into the nucleus. The transcription factor can then bind to its DNA target and regulate transcription.

If the targeted effector carries more than one effector moiety, they may all be initially trans-acting, or may all be initially cis-acting, or some may be trans- and some may be cis-acting. Furthermore, some or all of the effector moieties may be initially both cis- and trans-acting.

The effector moiety E may be, or may include, a molecule which induces the destruction or inactivation of the targeted effector once the desired intracellular modulation has been carried out. Such an effector moiety may be used to prevent long-lived recognition molecules from causing undesirable interactions within the cell. An example of an effector moiety which is capable of modifying the target molecule and of destroying the recognition moiety is ubiquitin ligase.

According to a further variant of the invention, the effector moiety is a molecule other than a transcription activator.

Particularly advantageous embodiments of the invention are the <<modifier>>, <<transporter>> and “forced interactor” embodiments. These variants will be described below.

The modifier embodiment of the invention involves the use of targeted effectors to bring about a change, particularly the creation of a covalent bond, in an intracellular target. It preferably involves the use of effector moieties which cannot normally act on that target, but which acquire the capacity to do so by virtue of their link to the recognition moiety. In other words, this variant preferably comprises the specific modification of an intracellular target by effector moieties of redirected specificity.

More specifically, accoding to the modifier embodiment, the effector moiety E has the capacity to exert an effect on the target T in a cell when it is covalently bound to the recognition moiety, but cannot exert this effect on T or C in the same cell in the same temporal and spatial environment when it is not covalently bound to the recognition moiety. Preferably, according to the <<modifier>> variant of the invention, E is initially trans-acting.

According to this embodiment, the inability of the effector moiety E to exert an effect on T or C in the cell when it is not covalently bound to the recognition moiety is generally due to the inherent specificity of E or T, for example in the case of an enzyme, whose inherent specificity prevents the target protein T from acting as substrate for E when it is not covalently linked to the recognition moiety.

Alternatively, the effector moiety E may be initially incapable of exerting an effect on T or C in the cell on account of a temporal or spatial specificity of T and/or E which prevents E and T from associating with each other when E is not covalently linked to the recognition moiety. Likewise, if E and T or C are not co-expressed in the cell, E will not be able to exert an effect on T and/or C.

The covalent association of the effector moiety E and the recognition moiety R permits the initial inability of E to act on T, to be overcome.

Another possibility is that the effector moiety E loses its ability to exert its effect on M when it is covalently bound to the recognition moiety.

According to the transporter embodiment of the invention, the effector moiety E has the initial capacity to transport an intracellular molecule M in cis to a predetermined sub-cellular compartment. On covalently joining E to R, E acquires the capacity to direct the target molecule T to the said sub-cellular compartment. According to this embodiment, E is therefore a transporter molecule or an addressing signal, such as NLS, secretory signal etc. The covalent association of the effector moiety and the recognition moiety enables E to exert this effect in trans on the recognition moiety's target T.

Preferably the sub-cellular compartment to which the target is transported by the effector moiety is one to which the target would not normally be addressed or to which it would only be addressed in specific conditions, or to a very limited extent. The transport is thereby <<forced>> by the targeted effector.

According to the “forced interactor” variant of the invention, oligomeric, particularly dimeric targeted effectors are used to provoke interactions between two targets which normally do not interact with each other at detectable levels.

The ability to bring close to one another two proteins that normally do not interact may allow them to interact. In the situation where one of the two proteins carries an enzymatic activity, it is conceivable that this activity could be exerted on the other protein that is normally not a substrate. The ability to induce interactions can be used to elucidate the biological function of the said interactions, and to modify cellular function.

By creating new interactions, the dimeric molecules of the invention augment the range of manipulations that can be exerted on molecular regulatory networks.

More specifically, the oligomeric intracellular recognition molecule of the invention, comprise from two to four, preferably two, intracellular recognition molecules R as defined above, said recognition molecules being covalently linked to each other, either directly or via a linker.

Preferably, but not necessarily, each of the monomers composing the dimer interacts with its target with an affinity corresponding to a Kd of less than 1×10^{8.} The dimer generally has a binding affinity of the same order as that of the component monomers.

Peptide aptamer dimers are particularly preferred. According to this preferred embodiment, the dimeric intracellular recognition molecule of the invention comprises two covalently linked intracellular recognition molecules R₁ and R₂,

wherein R₁ and R₂ each comprise a recognition domain V, V being a peptide having a length of five to sixty amino acids, conformationally constrained by covalent bonding to a platform,

R1 having the capacity to specifically interact, within a cell, with a site on a predetermined intracellular target molecule T1, and

R2 having the capacity to specifically interact, within a cell, with a site on a predetermined intracellular target molecule T2,

wherein R1 and R2 may be the same or different and T1 and T2 are distinct.

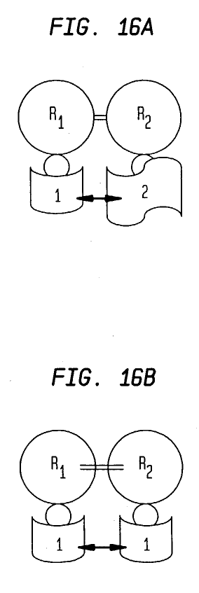

According to a preferred embodiment, the monomeric units of the dimer R1 and R2, are identical. and the intracellular recognition molecule specifically and simultaneously binds to two identical but separate target molecules T1 and T2. This is thus a homodimer interacting with two separate, normally non-interacting, identical targets. The forced interaction brought about by the dimer can lead to effects such as clustering of the target molecules (or non-covalent “dimerisation”). This type of dimer is illustrated in FIG. 16B.

According to a another preferred embodiment, the monomeric units of the dimer R1 and R2, are different, and the intracellular recognition molecule specifically and simultaneously binds to two different and separate target molecules T1 and T2. This is thus a heterodimer interacting with two separate, normally non-interacting, non-identical targets. The forced interaction brought about by the dimer can lead to effects such as clustering of the target molecules (or non-covalent “dimerisation”), or any of the effects listed above in connection with effector moieties in general. This type of dimer is illustrated in FIG. 16A.

The monomeric units of the aptamer dimers are covalently linked to each other either by direct fusion, or via a flexible linker. Examples of suitable linkers are given in the examples below.

The inventors have noted positional effects associated with different dimmer configurations. For example, in some situations, it has been observed that an aptamer in a dimeric structure binds its target more-tightly when it is in an N-terminal position. The configuration of the dimers can be designed taking this into account (see Examples below).

The dimers can be introduced into a cell already in their dimeric form, of alternatively can be formed within the cell for example by using leucine zippers etc.

The invention also relates to a method for preparing and identifying dimeric aptamers having the desired “forced interaction” capacities. More specifically, this aspect of the invention relates to a process for the identification of dimeric recognition moieties having the capacity to force an interaction between two target molecule T1 and T2, and/or a cellular component C which interacts directly or indirectly in a cell with T1 or T2 said process comprising the steps:

a) production of a random pool of peptides, so-called recognition moieties R,

b) screening of the random pool produced in (a) against T1 and T2 in a cell, in conditions suitable to allow identification of recognition moieties R capable of interacting with T1 or T2,

c) optionally contacting the moieties selected in (b) with proteins other than T1 and T2 to determine the specificity range of each of said moieties, and to identify moieties having a desired specificity range,

d) covalent linkage of two thus-identified recognition moieties R1 and R2 to each other to provide a dimeric recognition moiety,

e) screening of the dimeric recognition moiety to identify moieties capable of interacting simultaneously with T1 and T2.

Turning now to the intracellular target molecule T, this molecule can be any intracellular molecule. It may be endogenous to the cell or may be heterologous to the cell, for example a recombinant protein. It may also be a transmembrane molecule.

T may be a protein, a peptide, a nucleic acid, a carbohydrate, a phosphorylated molecule, a lipid, or a combination of any of these, for example a protein/DNA complex. The target T may be a molecule which is present only under certain conditions in the cell, for example a protein which is expressed with spatial or temporal specificity. T may be present in any or all cellular compartments, for example cytoplasmic or nuclear or uniformly distributed throughout the cell.

In some instances, the target protein T may be a protein comprising at least two functionally distinct protein domains, Tₐ and T_{b}, for example a synthetic fusion protein produced artificially in the cell, or a naturally occurring endogenous protein having distinct functions mapping to distict domains. In such a case, it is possible that the recognition moiety R of the invention specifically binds to a site on the first functional domain Tₐ and the effector moiety E exerts a structural or functional modulatory effect on the second functional domain T_{b}. This is particularly advantageous in cases where conservation of one of the functions of the target T is desirable, whilst modifying others.

The intracellular molecule upon which the targeted effector exerts its effect may in some cases not be directly the target molecule T, but a further intracellular component C with which T interacts, directly or indirectly. C may be any intracellular component, for example a protein, nucleic acid, lipid, phosphate, carbohydrate etc.

The interaction of the targeted effector with the target T or the intracellular component C gives rise to any one or more of a number of different chemical, biochemical, physical and/or functional changes. Examples of such modifications include:

chemical changes such as the formation or breakage of one or more covalent bonds or non-covalent bonds in T or in C,

physical changes such as the modification of the conformation of T or C, the transport of T or C to a sub-cellular compartment, or to the cell membrane on the inside or outside of the cell, labelling of T or C etc;

functional changes such as the enhancement or reduction in the efficiency of one or more functions of T or C, or the abolition or creation of a function;

The modulation of the chemical, physical, biochemical or functional properties of the target T or the cellular component C may give rise to a phenotypic change in the cell or organism. This phenotypic change may or may not be selectable or detectable.

The result of the modulation brought about by the targeted effector (including modifiers, transporters and forced interactors) may include one or more of the following effects:

homo- or heterodimerisation of T or C;

inhibition of homo- or heterodimerisation of T or C;

destruction of T or C;

stabilisation of T or C;

labeling of T or C;

ability of T or C to interact with a new partner;

localisation of T or C to a subcellular compartment to which T or C are not otherwise transported in the cell;

localisation of T or C to a subcellular compartment to which T or C are otherwise transported to a lesser degree in the cell;

exposure of T or C at the cell membrane;

internalisation of T or C;

provoking the association of T or C with natural intracellular partners;

prevention of the association of T or C with natural intracellular partners;

cleavage of T or C;

secretion of T or C.

According to a preferred embodiment of the invention, the recognition moiety R, in the absence of the effector moiety E, has no effect on the chemical, biochemical physical or functional properties of the target T i.e. R is neutral with respect to the the target protein T. In such cases, the modulation brought about by the targeted effector is entirely due to the effector moiety E. This type of interaction is possible, for example with peptide aptamers, because they interact with specific, clearly delimited sites or epitopes on a target. It is therefore possible to select aptamers which specifically interact with a desired target molecule T, but which do not happen to affect a particular function of T. As described above, if the aptamer is subject to mutagenesis of the variable region to optimise binding affinity, a slight modification of the selectivity of the aptamer can be provoked. Such controlled modification of the specificity may be used to create high affinity aptamers which are neutral with respect to the function of the target T.

It is also possible, according to the invention, that the interaction of the recognition moiety R with the target T give rise to the modification of the function of T. In such a case; the net result of the effector moiety on the target T is the combined action of the recognition moiety and that of the effector moiety.

According to a further variant, the effector moiety E does not affect the function of the target T or of the component C. In such a case, E may for example simply provide a label for the target.

The process of the present invention is carried out in a cell. The cell may be prokaryotic or eukaryotic. Eukaryotic cells are particularly preferred, for example, mammalian cells, including human and non-human cells, insect cells, plant cells, fish cells, avian cells, yeast cells etc. The cells may be cells in culture in vitro, or may be in whole organisms in vivo, or may be taken from a whole organism and treated ex vivo.

The targeted effector may be introduced into the cell in a number of different ways. For example, it can be introduced into the cell by expression of a DNA sequence encoding the targeted effector in the form of a fusion protein composed of the effector moiety and the recognition moiety. This method is generally applied when genetic manipulation of the cell or the organism is possible, and when the targeted effector is a protein. In such cases the targeted effector is introduced into the cell in a suitable vector, comprising all the necessary control sequences for expression.

Alternatively, the targeted effector is introduced into the cell in purified form using a cell permeable agent, such as protein transduction domains (PTDs), for example penetratin. This method is particularly advantageous for therapy when genetic modification of the individual is undesirable. A further alternative is to microinject the targeted effector into the cell.

The invention also relates to a method of producing the high affinity intracellular targeting molecules.

In particular, this aspect of the invention concerns a process for the production of a targeted effector having the capacity to specifically modulate the properties of an intracellular target molecule T, and/or a cellular component C which interacts directly or indirectly in a cell with T, said process comprising the steps

a) production of a random pool of peptides, so-called recognition moieties R,

b) screening of the random pool produced in (a) against T in a cell, in conditions suitable to allow identification of recognition moieties R capable of interacting with T,

c) optionally contacting the moieties selected in (b) with proteins other than T to determine the specificity range of each of said moieties, and to identify moietes having a desired specificity range,

d) covalent linkage of the recognition moieties R to an effector moiety E, E being a molecule which initially has the capacity to exert a predetermined effect on at least one intracellular component M, step (d) being carried out either prior to step (b), or after step (e),

e) verification of the affinity A₁ with which the recognition moiety R interacts with T, or of the affinity A₂ with which the targeted effector, interacts with T,

f) if both of A₁ and A₂ correspond to Kd values greater than 1×10⁻⁸ M, alteration of the binding region of the effector moiety to adjust the binding affinity of the interaction between T and the selected moiety so that the Kd becomes less than 1×10⁻⁸ M.

Step (b) in the above process may comprise the screening of the pool of random peptides in a cell against an unknown or known endogenous target molecule T, wherein interaction with T gives rise to a detectable or selectable phenotypic change. If approriate, the identity of T may then be determined by screening selected recognition moieties against molecules known to have the capacity to give rise to said phenotypic change, and deducing the identity of T. This method of producing and characterising targeted effectors can be employed when the nature of the effector moiety prevents the use of classic two-hybrid interaction trap system. Alternatively, the identity of T may be determined by using R or the targeted effector R-E as a <<bait>> in a two-hybrid interaction assay, and fishing potential <<T's>> from a library.

Alternatively, step (b) may comprise the screening of the pool of random peptides in a cell against a known artifical target molecule T, interaction with T giving rise to a detectable or selectable change. An example of this type of screening is the two-hybrid assay and its derivatives. According to this method, step (b) involves the detection of reporter gene transcription, artificial candidate target proteins being co-expressed in the cell with the recognition moieties.

The invention also relates to the targeted effectors themselves. More particularly, this aspect of the invention relates to a chimeric molecule comprising

a recognition moiety R, having the capacity to specifically interact, within a cell, with a site on an intracellular target molecule T, the interaction with T occurring with an affinity A₁, and

an effector moiety, E, covalently linked to R, E being a molecule which has an initial capacity to exert an effect on at least one molecule M, and which when it is covalently linked to R, acquires the capacity to specifically exert the effect on the intracellular target molecule T,

wherein the targeted effector interacts with T with an affinity A₂, the affinity Al or the affinity A₂ corresponding to a K_{d} value of less than 1×10⁻⁸ M.

Preferably, the binding affinity Al is less than or equal to 1×10⁻⁹ M, for example comprised between 1×10⁻⁹ and 1×10⁻¹³ M, for example less than 1×10⁻¹⁰ or less than 1×10⁻¹¹ or less than 1×10⁻¹² M.

The invention also relates to the high affinity <<naked>> recognition moiety <<R>>. This aspect of the invention concerns more specifically an intracellular recognition molecule R, composed of a conformationally constrained recognition domain, displayed in a platform,

said recognition molecule having the capacity to specifically interact, within a cell, with a site on a predetermined intracellular target molecule T, the interaction with T occurring with an affinity corresponding to a K_{d} value of less than or equal to 5×10⁻⁹ M.

The naked recognition moiety R preferably interacts with T with an affinity corresponding to a K_{d} value comprised between 1×10⁻⁹ M and 1×10⁻¹³ M, for example between 1×10⁻⁹ M and 1×10⁻¹² M. The characteristics of the naked recognition moiety are described in detail above for <<R>>.

The invention also relates to a pharmaceutical composition comprising the targeted effector of the invetion, or a nucleic acid encoding such a targeted effector, in association with a pharmaceutically acceptable excipient.

The chimeric targeted effectors of the invention and the nucleic acids encoding the targeted effectors can be used in therapy. For example, the targeted effectors or the nucleic acids can be used in methods of treatment, or in the preparation of medicaments for treating

microbial infections, including viral infections, and fungal infections;

immunological disorders;

neurological disorders;

metabolic disorders;

psychiatric disorders;

myopathies;

genetic disorders;

cancer;

cardiovascular disorders;

dental disorders.

Preferred embodiments of the invention, which will be described in detail in the Examples below, are peptide aptamer derivatives which covalently modify and change the localization of target proteins in vivo. In accordance with the invention, the peptide aptamers were made by first generating a higher-affinity mutant for example an anti-Cdk2 aptamer, mutagenizing the variable region of an existing aptamer and screening for tighter binding mutants using relatively insensitive two-hybrid reporter genes. This variant had a significant increase in affinity and exhibited a Kd between 2 and 5 nM. Use of still-less sensitive reporter genes (28), and/or LexA mutants with a decreased affinity for their operators (29) and/or substitution of weaker activation domains on the aptamer library, should allow the identification of mutant aptamers with sub-nanomolar affinity in one step.

To construct modifiers, proteins that covalently alter target proteins in vivo, ubiquitination i.e. the coupling of ubiquitin molecules to lysine residues on proteins, was exploited. These modifiers ubiquitinated the LexA-Cdk2 target. Moreover, the results suggest that the modifiers destroyed themselves, by self-ubiquitination on lysine residues exposed at the surface of the thioredoxin platform. This observation is consistent with the fact that the native Rsp5 protein ubiquitinates itself in vitro, probably on lysine residues lying in the amino-terminal part of the protein, outside of its hect domain (23,30).

The invention provides the first in vivo demonstration of targeted protein modifications by enzymes of redirected specificity. However, these aptamer-hect fusions did not destroy their Cdk2 targets, even those that contained extra lysine residues. It is possible that, for Cdk2, ubiquitination mediated by a more active effector domain would result in destabilization. Consistent with this idea, Gosink et al. redirected the specificity of two plant E2 ubiquitin-conjugating enzymes, Ubc1 and Ubc4, in vitro by fusing different protein-binding peptides, including the Ig binding domains of Staphylococcus aureus A protein, to Ubc carboxy-termini (31). In vitro, these authors observed ubiquitination of the cognate substrates, and a partial degradation of the targeted IgG. However, the most likely explanation for the stability of ubiquitinated LexA-Cdk2 fusions is that Cdk2 is simply refractory to ubiquitin-mediated proteolysis. In fact, no Cdk is known to undergo ubiquitin-dependant proteolysis, although some of its binding partners are degraded by this means (32). A number of other proteins are also ubiquitinated without being degraded, including H2A (24), cyclins in certain cell cycle phases (33) and some membrane receptors whose ubiquitination signals endocytosis without involving the proteasome (34).

Peptide aptamers fused to a nuclear localization sequence were also used as “transporters”. Anti-Cdk2 and anti-Ste5 aptamers that carried nuclear localization signals caused their targets to accumulate in the nucleus. The results suggest that it will be possible to build transporters with other protein moieties that readdress their protein targets to other subcellular compartments: the endoplasmic reticulum (36), the mitochondrial membrane (37) or the plasma membrane (38). The aptamers in these transporters all blocked the activity of their target proteins, but, as has been noted in the case of the NLS containing anti-Ste5 aptamers, inactivation may in fact be caused by enforced nuclear localization, rather than by blocking interaction with partners (7). Because transporting aptamers allow mislocalization of targeted proteins that are expressed under the control of their own promoters, the perturbations that transporters induce in cell function should be less severe than those caused by overexpression of target proteins fused to addressing sequences.

The construction of new proteins from different functional modules has been reported for many types of proteins (28, 39), and the success of these methods can be taken to support the current picture in which exons are shuffled among proteins during evolution (40). Peptide aptamers represent useful general purpose recognition moieties in chimeric proteins with other effector domains with other functions. The ability to control the spatial and temporal expression of such “modifiers” and “transporters” in cells and whole organisms should facilitate a finer control of protein modification, inactivation, and localization. Furthermore, the ability to select aptamers that distinguish among allelic variants of proteins should allow selective modification of the activities of individual alleles. The generation and use of new peptide aptamer derivatives should facilitate high-resolution study of regulatory pathways, and could be useful in new therapeutic strategies

A further embodiment of the invention relates to specific targeted effector molecules, preferably aptamers, which have the capacity to specifically interact with a target chosen from a cyclin-dependent kinase and a pro-apoptotic protein.

Preferably, the cyclin-dependent kinase is Cdk2. Such an aptamer has a “V” sequence which is a mutant of the amino acid sequence QVWSLWALGWRWLRRYGWNM, said mutant having from one to five, preferably one to three amino acid changes with respect to said sequence. A particularly preferred anti-Cdk2 aptamer has a peptide recognition domain comprising or consisting of the amino acid sequence QVWSSWALGWRWLRRYGWGM.

As a preferred pro-apoptotic molecule, Bax can be cited. Anti-Bax aptamers of the invention have a peptide recognition domain comprising or consisting of a mutant of the amino acid sequence PRGAPMWMRWVCQMLETMFL, said mutant having from one to five, preferably one to three amino acid changes with respect to said sequence. Preferably, the peptide recognition domain comprises or consists of the amino acid sequence PRGAPMWLRCVCQMLETKFL.

Monomeric and dimeric aptamer constructs using these anti-Cdk2 and anti-Bax aptamers are described in the Examples below. Heterodimers of anti-Cdk2 and anti-Bax aptamers are particularly preferred.

Different aspects of the invention are illustrated in the figures:

Figure Legends

Table 1. GFP Interaction Phenotypes and Kds.

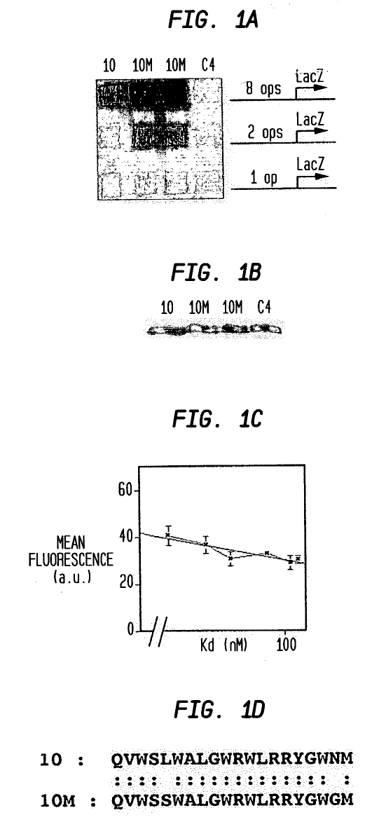

Average green fluorescence of yeast above background caused by interactions between LexA-Cdk2 and aptamers 2, 3, 5, 8, 10 and 11 in 4 independent experiments. Previously measured Kds (1) were plotted against measured fluorescence (FIG. 1c). The plot fit the following equation Kd=10 exp-(fluo+123.7/21.9). We calculated a 2 nM Kd for aptamer 10M by interpolation using this equation.

FIG. 1. A mutant aptamer with enhanced affinity for its target. (a) Interaction mating assay between LexA-Cdk2 and aptamer 10, two strains carrying aptamer 10M, and a non-interacting aptamer C4, using three different sensitivity LexAop-lacZ reporters. (b) Western blot assay using an anti-TrxA antibody. Diploid exconjugates were grown in galactose containing medium, and proteins were extracted and subjected to a Western blot analysis with anti-TrxA antibody. (c) Strength of interaction phenotypes as determined by fluorescence from a LexAop-GFP reporter plotted against Kds measured in evanescent wave experiments. Fluorescence values are in arbitrary units (a.u.). (d) Sequence of the variable regions of aptamers 10 and 10M.

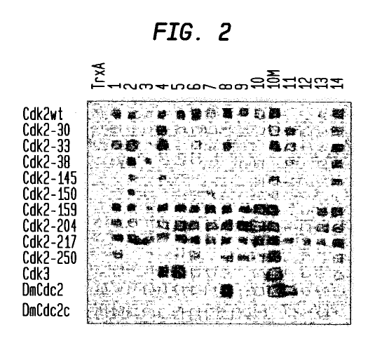

FIG. 2. Mapping sites on Cdk2 bound by the original aptamers and of aptamer 10M. The Cdk2 mutant bait proteins, described elsewhere (3), Cdk3, and Drosophila Cdc2 and Cdc2c (16) were collected. In this experiment, TrxA, the 14 different aptamers originally selected (1), and aptamer 10M were expressed as preys. Yeast was mated to generate an interaction matrix (16).

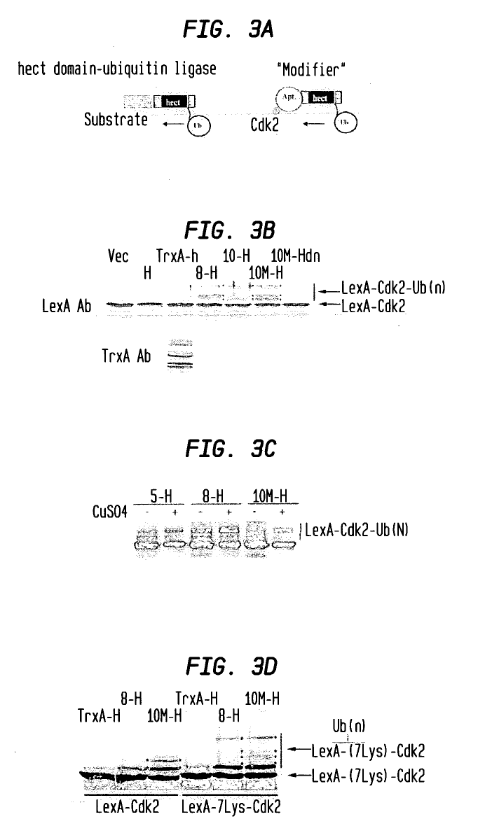

FIG. 3. Targeted ubiquitination of LexA-Cdk2 by aptamer-hect fusions. (a) Design of a “modifier”, inspired by the structure of hect domain containing ubiquitin ligases. (b) Western blot analysis of LexA-Cdk2 (upper panel) and TrxA-hect or aptamer-hect fusions (lower panel) using anti-LexA and anti-TrxA antibodies respectively. (c) Western analysis of LexA-Cdk2 when aptamer-hect fusions are expressed by growth overnight in a medium that does or does not contain CuSO4 and that does or does not express Myc-tagged ubiquitin. (d) Western blot analysis of LexA-Cdk2 and LexA-7Lys-Cdk2 when TrxA-, 8- and 10M-hect fusions are expressed, using the anti-LexA antibody.

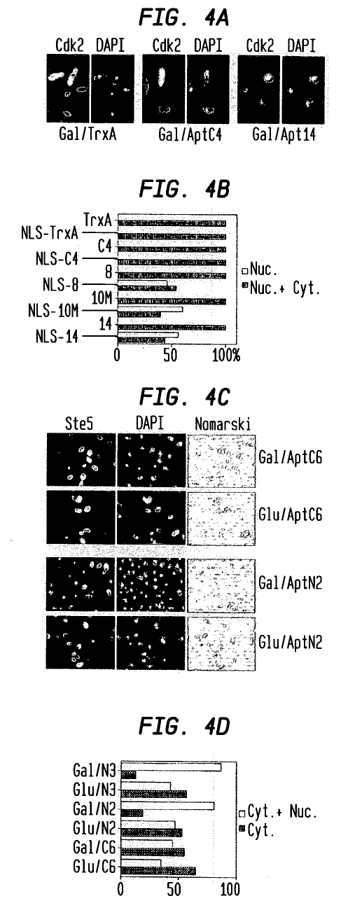

FIG. 4. Nuclear translocation of LexA-Cdk2 and LexA-Ste5 by interacting NLS-aptamer derivatives. (a) Yeast photomicrographs. Left panels: Indirect immunofluorescence of LexA-Cdk2 protein using anti-LexA antibody. Right panels: DNA staining with DAPI. Gal/TrxA: TrxA is expressed. Gal/AptC4: control aptamer C4 is expressed. Gal/Apt14: anti-Cdk2 aptamer 14 is expressed. (b) Percentage of yeast that displayed clear nuclear immunofluorescence, in presence of aptamers addressed to the nucleus or not. Dark bars: nuclear and cytoplasmic staining. White bars: nuclear staining. At least 50 cells were observed for each assay. (c) Yeast photomicrographs. Left panels: Indirect immunofluorescence of LexA-Ste5 fusion protein using anti-LexA antibody. Center panels: DNA staining with DAPI. Right panels; yeast observed with Nomarski optics. Gal/AptC6: non-NLS aptamer C6 is expressed. Glu/C6: non-NLS aptamer C6 is not expressed. Gal/AptN2: NLS-aptamer N2 is expressed. Glu/N1: NLS-aptamer N2 is not expressed. (d) Percentage of yeast that displayed cytoplasmic+nuclear, or purely cytoplasmic staining, in presence of various aptamers addressed to the nucleus or not. Dark bars: cytoplasmic staining. White bars: cytoplasmic and nuclear staining. At least 100 cells were observed for each assay.

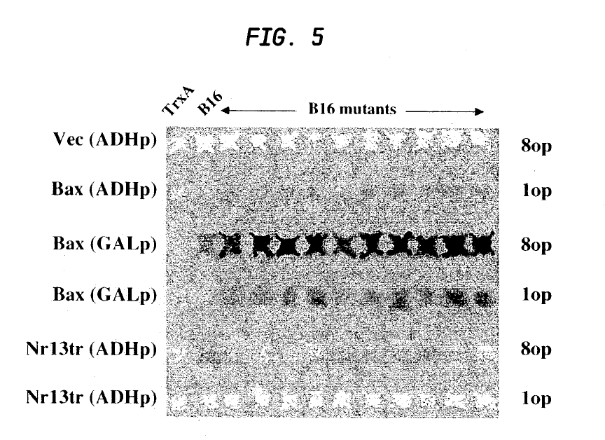

FIG. 5: Affinity of aptamers evolved in vitro. This matrix shows the improved affinity of the 10 mutants as compared to the parental aptamer (B16). On the left, the bait proteins and the promoters used to express them are indicated. On the right, the lacZ reporter genes are indicated. Considering the respective strengths of the ADH and GAL promoters on galactose medium, the sensitivity of the 2H assay can be ranked as follows, from the least to the most sensitive configuration

ADHp/1op

GALp/1op

GALp/8op This matrix clearly shows that all mutants bind Bax more tightly than B16. It also shows that while some mutants may bind Nr13 more tightly, others no longer bind Nr13, at least at a detectable level.

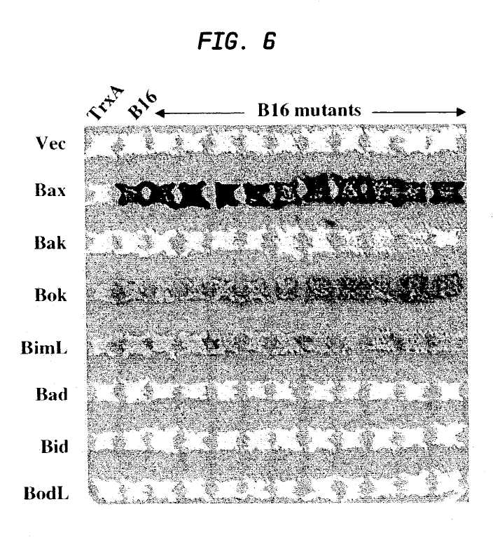

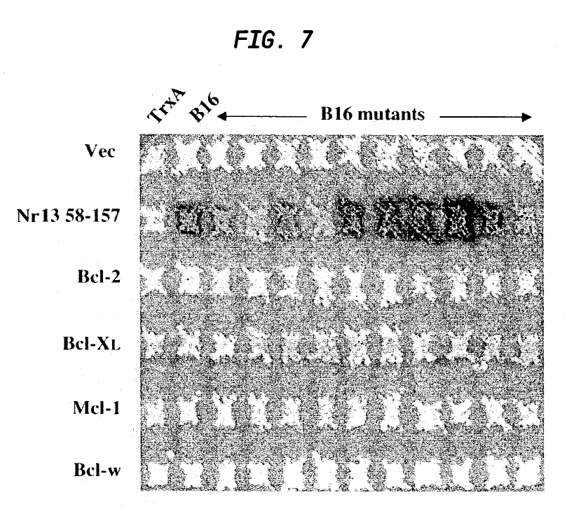

FIGS. 6 and 7: Specificity of aptamers evolved in vitro. FIG. 7 confirms the observation made in FIG. 5. None of the mutant aptamers bind anti-apoptotic proteins (Bc1-2, Bc1-X1 etc . . . ), other than Bax, at detectable levels, like the parental aptamer. FIG. 6 shows pro-apoptotic proteins (Bax, Bak, Bok, etc . . . ). It can be seen that most, if not all mutants do not bind any of these proteins, like the parental aptamer. Two mutants may have acquired the ability to bind to Bok, although this cannot be determined unequivocally since Bok expressed as a bait protein activates transcription of the reporter gene per se.

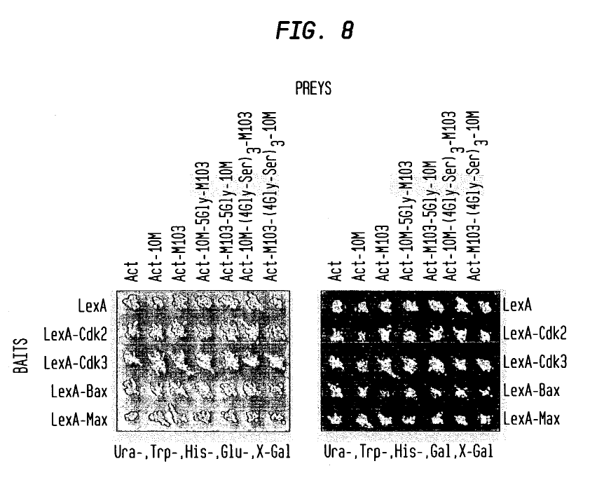

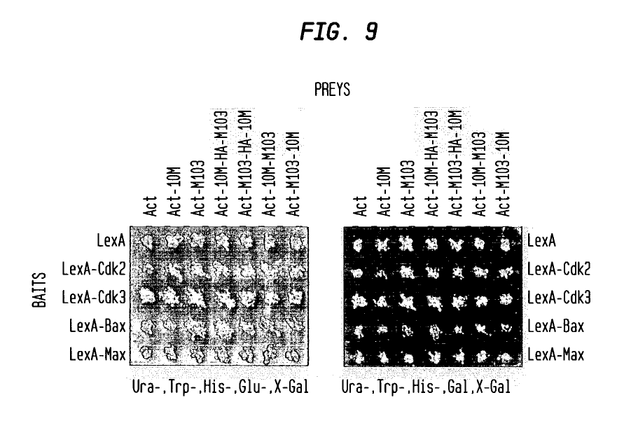

FIGS. 8 and 9: Two-hybrid interaction mating assays to detect binding of aptamer dimers, consisting of an anti-Cdk2 (10M) and an anti-Bax aptamer (M103), to target proteins Cdk2 and Bax. FIG. 8 illustrates the matrix obtained with dimers having 5Gly and (4 Gly-Ser)ₓ linkers. FIG. 9 illustrates the matrix obtained with dimers having HA or no linkers. Baits fused to DNA binding domain LexA are shown horizontally. Aptamer preys fused to transcription activator Act are shown vertically. Composition of culture media is indicated below the left and right panels.

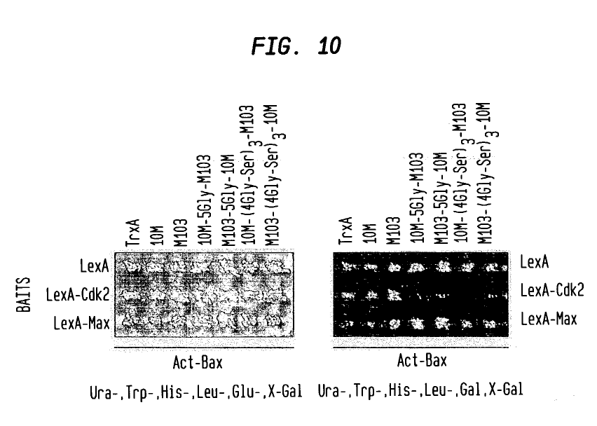

FIG. 10: Interaction assay using a three-component system to detect forced interactions between target molecules, induced by aptamer dimers. Baits fused to DNA binding domain LexA are shown horizontally. The prey protein consists of Bax fused to transcription activator Act (expressed in all strains streaked vertically). Aptamer dimers, susceptible of acting as bridges between the bait and the prey, are shown horizontally above the panels. Composition of culture media is indicated below the left and right panels. Transcription of the reporter gene, providing evidence of interaction between bait and prey, is seen as dark coloured yeast.

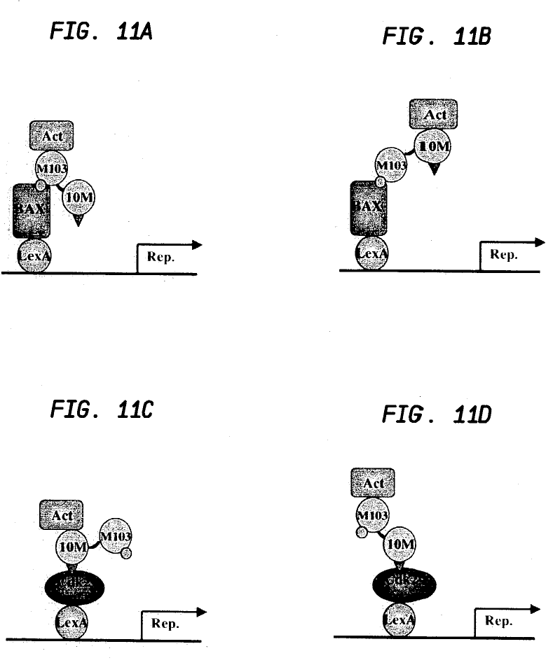

FIG. 11: schematic representation of a two-hybrid assay for dimer binding (as shown in FIGS. 8 and 9). Aptamer preys are fused to transcription activator Act. Baits comprise either Bax (FIGS. 11A and 11B) or Cdk2 (FIGS. 11C and 11D) fused to DNA binding domain LexA. The four panels A, B, C and D represent the different possible configurations depending upon the position of the anti-Bax (103M) or anti Cdk2 (10M) aptamer within the dimer.

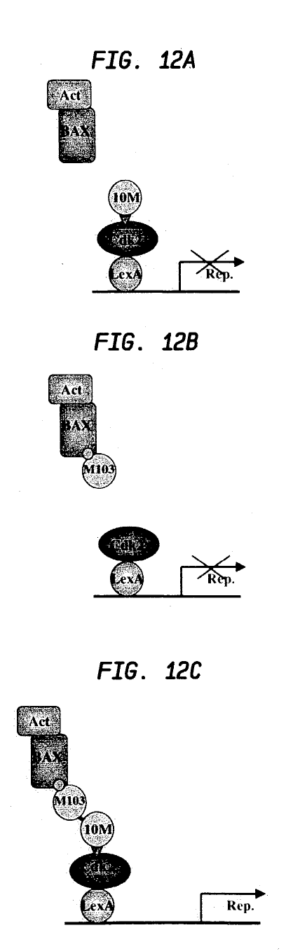

FIG. 12: schematic representation of a two-hybrid assay for forced interaction between two target proteins Cdk2 and Bax, brought about by an aptamer dimer (as shown in FIG. 10). Panels A and B show absence of forced interaction when a monomeric aptamer against Cdk2 (Panel A) or against Bax (Panel B), is used. Panel C shows forced interaction between Cdk2 and Bax brought about by the aptamer dimer M103-10M.

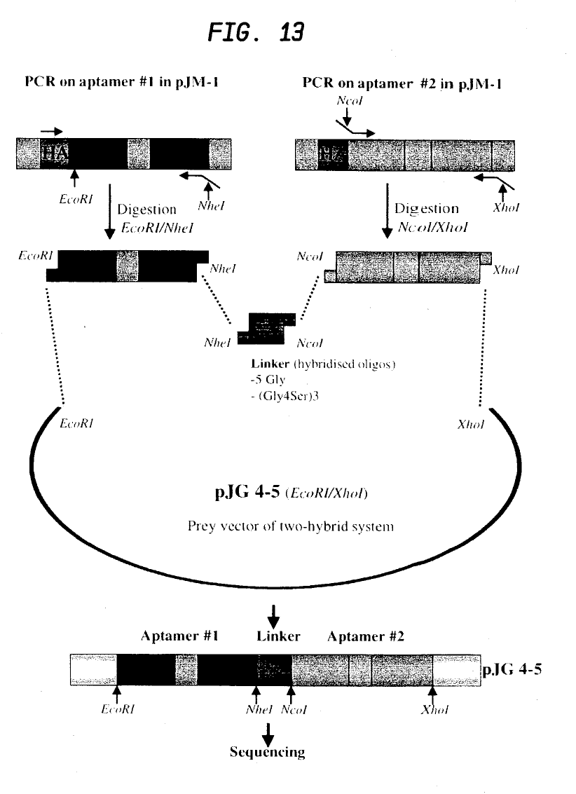

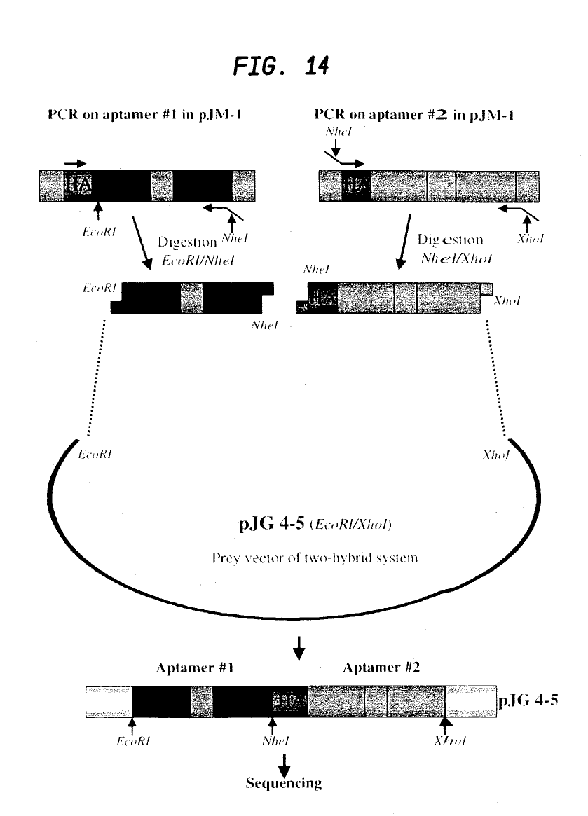

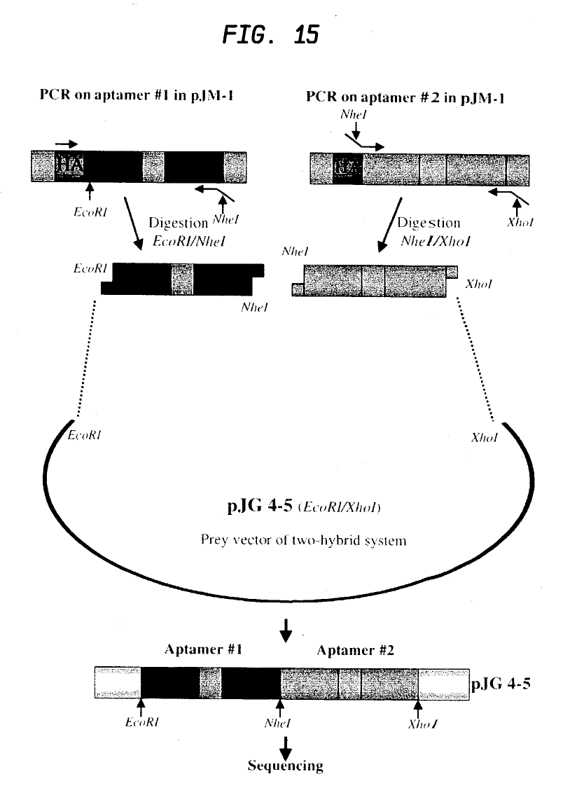

FIGS. 13, 14 and 15 construction of Aptamer dimers. FIG. 13: Linkers of the 5-Glycine and (4-Glycine 1 Serine ) X3 type; FIG. 14 Linkers of the HA type; FIG. 15: Direct fusion of the monomeric aptamers, without an intervening linker molecule.

FIG. 16: schematic representation of an aptamer heterodimer (panel A) and homodimer (panel B) according to the invention. A: Bispecific aptamer dimmer. Aptamer R₁, comprising a display platform shown as a large circle, and a variable region shown as a small circle, specifically recognises an epitope on a first Protein 1. Aptamer R₂, specifically recognises an epitope on a second Protein 2.

B: Monospecific monoparatopic aptamer dimer. Aptamer R₁ specifically recognises an epitope on a first Protein 1. Aptamer R₂ is identical to Aptamer R₁. The dimeric aptamer thus has the capacity to specifically recognise two discrete Protein 1 target molecules.

Double lines represent a covalent bond between the aptamers. A double-headed arrow represents an interaction between target molecules 1 and 2, brought about by the Aptamer dimer

### EXAMPLES

In the following examples, derivative peptide aptamers are described that covalently modify or change the subcellular localization of their target proteins. First, selection of a peptide aptamer with an improved affinity for its target is described. This improved aptamer and others are used to construct two types of chimeric proteins: “modifiers”, which ubiquitinate their target proteins, and “transporters”, which change the subcellular localization of their targets.

Construction of aptamer dimers is also described, and their use in bringing about forced protein-protein interactions is demonstrated.

The materials and methods used in the following examples are described in detail in the section <<Materials and Methods>>.

Results

### Example 1

Generation and Identification of a Higher Affinity Anti-Cdk2 Aptamer

An existing anti-Cdk2 aptamer was mutagenized to select one with higher affinity. The starting molecule, aptamer 10, has a measured Kd of 1.05×10⁻⁷M (1). Random PCR mutagenesis was performed on the aptamer 10 V region, and the PCR products were reintroduced into the library vector, pJM-1. This vector directs the expression of aptamers fused to the SV40 nuclear localization sequence, the B112 activation domain, and the HA epitope tag under the yeast GAL1 promoter (1).

A pool of 15,000 mutants was created. To obtain a measure of the efficiency of the PCR mutagenesis, the V regions of two clones from this pool were sequenced; sequencing revealed that these carried 3 and 4 mutations that resulted in 1 and 3 amino acid changes respectively. To identify tighter-binding variants from this pool, advantage was taken of the existence of different LexA operator reporter genes with different sensitivities. A strain that expressed a LexA-Cdk2 bait and that carried pRB1840, a relatively insensitive lacZ reporter (13) with a single synthetic LexA operator was used initially. This reporter was not activated by aptamer 10 (FIG. 1a). The pool of PCR mutagenized plasmids was introduced into this strain, and transformants that gave rise to blue colonies were identified. The V regions of the 7 plasmids thus identified carried identical nucleotide changes, and thus were presumably not independent. The nucleotide changes resulted in two amino acid changes: Leu to Ser at V region position 5, and Asn to Gly at position 19 (FIG. 1d):

| | |
|---|---|
| Aptamer 10: | QVWSLWALGWRWLRRYGWNM |
| Aptamer 10M: | QVWSSWALGWRWLRRYGWGM |

This mutant did not interact with Max and Rb, proteins unrelated to the Cdk family (not shown). The mutant aptamer was termed <<10M>>.

The affinity of 10M and 10 for Cdk2 was compared by interaction mating. The reporters, pSH18-34, pJK103, and pRB1840, contain respectively 8 high affinity operators, 2 high affinity operators, and 1 lower affinity LexA operator (FIG. 1a). While, as judged by blue color from the pSH18-34 reporter, aptamer 10M had only a slightly greater affinity than aptamer 10, blue color from pJK103 and pRB1840 clearly indicated that 10M bound LexA-Cdk2 more strongly. To verify that the apparent higher affinity was not due to increased expression and/or stability of aptamer 10M, Western blotting experiments were performed and showed that the steady-state levels of aptamer 10, 10M, and a control aptamer were identical (FIG. 1b).

The gain in affinity was measured by two different means. First, a LexAop-GFP reporter gene was used to quantify interactions between anti-Cdk2 aptamers and LexA-Cdk2. Mean fluorescence obtained from each interaction was plotted against Kds measured in evanescent wave experiments (1). This plot followed a logarithmic equation (Table 1, and FIG. 1c). This equation was used to calculate the Kd of the interaction between LexA-Cdk2 and aptamer 10M from the fluorescence it conferred (Table 1). Evanescent wave experiments were also performed with purified aptamer 10M and His6-Cdk2 (12). The measured Kd was 5 nM (not shown), quite close to the 2 nM Kd calculated from the GFP data.

TABLE 1

| | | Mean Fluorescence | Standard |
|---|---|---|---|
| Aptamer | Kd (nM) | (arbitrary units) | deviation |
| 8 | 38 | 40.8 | 4.3 |
| 5 | 52 | 36.8 | 3.7 |
| 2 | 64 | 30.9 | 3.0 |
| 11 | 87 | 33.0 | 1.4 |
| 10 | 105 | 29.0 | 2.8 |
| 3 | 112 | 30.3 | 1.7 |
| 10M | 2 | 67.5 | 3.8 |

To evaluate the contributions of the V region changes to increased affinity, we mutated both residues individually to wild type and analyzed the single mutants by interaction mating (data not shown). These experiments showed that the mutation of V region residue 5 (Leu to Ser) contributed to the gain of affinity, but that the mutation residue 19 (Asn to Gly) did not.

In order to determine whether the gain in affinity in 10M was caused by changes in its contact(s) with Cdk2, binding of all existing anti-Cdk2 aptamers, including 10M, to a collection of Cdk2 mutants, related members of this protein family, and control proteins (not shown) was examined. FIG. 2 shows that, as previously observed, some of these aptamers recognize different epitopes conserved among Cdk proteins (1). However, by contrast with the aptamers isolated in previous work, aptamer 10M showed distinct crossreactivity to other Cdk proteins. This interaction with Cdk family members is consistent with three ideas. First, the Leu to Ser change might create direct contact(s) between the variable region and residue(s) conserved among the Cdk proteins tested. Second, the change might indirectly create contacts by changing the conformation of the variable region. Third, even though Leu is more dihedrally constrained than Ser, a change to Ser might create new internal contacts within the variable region and thus reduce its conformational entropy.

### Example 2

Targeted Intracellular Protein Modifiers

Aptamer 10M and others were used to make protein derivatives that ubiquitinated target proteins in vivo. The design was based on the structural organization of the hect domain-containing ubiquitin ligases, which conjugate ubiquitin received from a E2 enzyme and transfer it to a protein substrate (22). The amino-terminal substrate-recognizing region of these enzymes varies greatly, whereas the carboxy-terminal region, the hect domain, which carries the catalytic activity, is conserved between family members and throughout evolution (23). This modular structure suggested that we could fuse a hect domain to peptide aptamers and create ubiquitin ligases with engineered specificities (FIG. 3a).

Accordingly, a hect domain native to the yeast protein Rsp5 was used to construct various aptamer-hect fusions. These were exprssed in yeast together with their putative LexA-Cdk2 targets.

The specificity of the aptamer-hect fusions, as evaluated indirectly by the capacity to ubiquitinate, was seen to reflect that observed in the interaction matrix generated in yeast assays using the corresponding aptamer without hect. The aptamer-hect fusions were able to discriminate between closely related target molecules such as Cdk2 and Cdk3.

The fates of these fusion proteins was examined. FIG. 3b shows that while the control TrxA-hect fusion that lacked a variable region was detectable by Western analysis, none of the anti-Cdk2 aptamer-hect fusions could be detected. This result indicates that attachment of a TrxA aptamer to a hect domain destabilizes the aptamer. However, expression of anti-Cdk2 aptamer-hect fusions resulted in the appearance of a ladder of higher molecular weight forms of LexA-Cdk2, suggesting that these chimeric proteins, even expressed at low levels, still caused ubiquitination of the target. The ladder of higher molecular weight LexA-Cdk2 forms was most apparent when the 10M-hect fusion was expressed, suggesting that the affinity of the modifier for the target affected the degree of target modification. Modification did not result in destruction: as determined from Western (FIG. 3b) and pulse chase experiments (not shown), expression of these modifiers had no effect on LexA-Cdk2 stability or half life.

To confirm that this ladder of higher molecular weight species corresponded to LexA-Cdk2-ubiquitin conjugates, two different experiments were performed. First, a yeast strain was used that conditionally expressed Myc-tagged ubiquitin, under the control of a copper-inducible promoter (15), and repeated the above experiments. This ubiquitin derivative is larger than native ubiquitin, and conjugates that contain it have higher molecular weights (15). On addition of CuSO4 to the culture medium, the bands on the ladder of higher molecular weights shifted higher than those in the uninduced culture (FIG. 3c). Second, a loss of function anti-Cdk2 aptamer-hect fusion was generated by changing the cysteine residue that forms the thioester bond with ubiquitin to alanine (23). In cells that expressed this mutant fusion, we did not observe the LexA-Cdk2 ladder, but now did observe, for the first time, the enzymatically-dead aptamer-hect derivative (FIG. 3b). This fact suggests that active aptamer-hect derivatives may be sensitive to proteolysis after self-ubiquitination. This idea is supported by two lines of evidence. First, ubiquitination occurs on lysines (24), and, by contrast with aptamer 5-hect, 8-hect, 10-hect, and 10M-hect fusions, aptamer 2-hect and 11-hect fusions, which contain lysines in their V regions, did not ubiquitinate LexA-Cdk2 (not shown), even though they bind Cdk2 as tightly as the others (FIG. 2). This fact is consistent with the idea that ubiquitination of their V regions blocks their binding. Second, variant aptamer-hect fusions in which we changed different combinations of the 5 solvent-exposed lysines (K19, 37, 53, 70, 83) (25) on TrxA to arginines resulted in proteins detectable by anti-TrxA antiserum (data not shown). These observations suggest that the aptamer-hect fusions are vulnerable to ubiquitination by their own hect moieties and subsequently proteolyzed, but are able to ubiquitinate their targets even though expressed at very low steady states.

Finally, it was tested whether LexA-Cdk2 could be sensitized to ubiquitin-dependent proteolysis by introducing into it extra lysines as ubiquitin acceptors. To this end, a LexA-Cdk2 derivative that carried 7 lysines between the LexA and the Cdk2 moieties was expressed in yeast. This LexA-Lys7-Cdk2 target was co-expressed together with TrxA-hect, 8-hect, and 10M-hect modifiers and was visualized with anti-LexA antibody as above. FIG. 3d shows that, as compared with the “native” LexA-Cdk2 target, the additional lysines on the experimental target improved its ubiquitination, both increasing the amount of higher molecular weight LexA-Cdk2 derivatives, and causing the appearance of at least one new still-higher molecular weight conjugate. However, as judged by its steady-state level, the increased ubiquitination of this LexA-Lys7-Cdk2 target did not destabilize it (FIG. 3d).

### Example 3

Targeted Intracellular Protein Transporters

The possibility of using derivatized aptamers to change the localization of their protein targets in vivo was investigated. In the original library vector, aptamers are expressed in yeast fused to an SV40 nuclear localization sequence (NLS). LexA-fusion proteins that lack nuclear localization signals are uniformly distributed within the yeast cell (26, this study). It was tested whether anti-Cdk2 aptamers addressed to the nucleus would also concentrate LexA-Cdk2 in the nucleus. As shown by immunofluorescence experiments, LexA-Cdk2 is uniformly distributed in cells in which the chimeras B112-NLS-TrxA and B112-NLS-aptamer are not expressed. Similarly, LexA-Cdk2 is evenly distributed inside cells in which the control chimera B112-NLS-TrxA is expressed. However, in cells in which B112-NLS-anti-Cdk2 aptamers are expressed, LexA-Cdk2 is concentrated in the nucleus (FIG. 4a). All the tested fusions triggered substantial nuclear localization of their LexA-Cdk2 target.

To confirm that the concentration of LexA-Cdk2 in the nucleus was due to the nuclear translocation of peptide aptamers rather than any other aspect of the binding of aptamers to their target, LexA-Cdk2 was expressed together with either aptamers or NLS-aptamers and determined the percentage of yeast in which LexA-Cdk2 was clearly nuclear. The results show that the nuclear localization of LexA-Cdk2 depends on the expression of peptide aptamers that contain nuclear localization sequences (FIG. 4b). Finally, NLS-containing aptamers made against another protein, Ste5 (7) were used to determine whether NLS-aptamer fusions could cause nuclear localization of a protein that is thought to be to be predominantly cytoplasmic (27). When no aptamer was expressed or when a non-nuclear localized aptamer was expressed, LexA-Ste5 showed a predominant cytoplasmic localization. However, when NLS-aptamers were expressed, LexA-Ste5 showed a distinct concentration in the nucleus (FIGS. 4c, d)

### Example 4

Generation and Identification of Higher Affinity Anti-Bax Aptamers:

An experiment similar to those described above for anti-CdK2 aptamers was carried out using aptamer <<B16>>, a Bax-interacting peptide aptamer that also recognises Nr13tr, a protein belonging to the same functional family.

The structure of the B16 aptamer was the same as those described earlier (ref. 1). Aptamer B16 carries an NLS and an Act moiety for transcriptional activation of a reporter gene in the two hybrid interaction assay.

Mutagenesis of the variable domain was carried out using mutagenic PCR as described above. Ten B16 mutants were selected for further studies.

The results of the affinity and specificity matrices are shown in FIGS. 5, 6 and 7. All three figures show interaction matrices, where yeast strains expressing bait proteins and conatining Lac Z reporter genes are streaked horizontally and yeast strains expressing aptamers are streaked vertically.

In contrast to the CdK2 experiments, here, aptamers having a stronger affinity to Bax, and an unchanged specificity, or even a sharper affinity towards their target, could be generated.

### Example 5

Dimers of Peptide Aptamers:

Aptamer dimers have been designed to bring into proximity two proteins, Cdk2 and Bax, that do not normally interact. High affinity binding of the dimer to its target proteins is shown to provoke measurable interaction between Cdk2 and Bax in two-hybrid assays.

a) Design of Dimers

The aptamer dimers exemplified here consist of the fusion of two peptide aptamers within the same polypeptidic chain. These two aptamers are either separated by a peptidic linker or are directly joined to each other without a linker (see Materials and Methods, and FIGS. 13-15). The following four different types of intervening sequences have been used as linkers:

1-5 Glycines

2-(4 Glycines−1 Serine)×3

3-The epitope tag HA

4-None

Linkers 1 and 2 above are well-known flexible sequences.

The two peptide aptamers that have been used here to establish the proof of concept are aptamer “10M” (see Example 1 above), and aptamer “M103” (see Materials and Methods), selected against Cdk2 and Bax respectively. These two proteins are not known to interact, and Bax is not a known substrate of Cdk2. Both aptamers have been selected through an in vitro evolution procedure, starting from interacting aptamers. While the affinity of 10M for Cdk2 has been measured (Kd=5 nM), the affinity of M103 for Bax has not been quantified but appears to be at least as good as that of 10M.

Eight dimers have been constructed, thereby totally covering the combinatorial of linkers and relative position of the two peptide aptamers (see table below, and FIGS. 13, 14 and 15).

| DIMER | POSITION 1 | LINKER | POSITION 2 |
|---|---|---|---|
| 10M:M103 | 10M | 5 Gly | M103 |
| | ″ | (4 Gly - 1 Ser) × 3 | ″ |
| | ″ | HA | ″ |
| M103:10M | ″ | None | ″ |
| | M103 | 5 Gly | 10M |
| | ″ | (4 Gly - 1 Ser) × 3 | ″ |
| | ″ | HA | ″ |
| | ″ | None | ″ |

b) Dimer Properties

i) Test for Dimer Binding:

As a first step in the investigation of dimer properties, it was determined whether the aptamers contained in the dimers have conserved the property of interaction with their cognate target protein (Cdk2 and Bax). This was indeed a prerequisite to the “forced interaction” activity sought.

To address this concern, two-hybrid assays were performed. The dimers were cloned into pJG4-5, a yeast vector directing the inducible expression of “preys”. Their ability to interact with Cdk2 and Bax was examined in an interaction mating assay (FIGS. 8 and 9). This assay established that the dimers interact individually with Cdk2 and Bax. Interestingly, the interaction phenotypes (as evaluated by the level of blue in the interaction matrix) appeared stronger when the interacting aptamer was in position 1 (i.e. N-terminal) of the dimer (especially observed when using Bax as the bait). At present, the reasons for this positional effect are not clear. A partial proteolytic cleavage at the junction between both aptamers was ruled out by western blot experiments (not shown).

It can also be seen that the apparent affinities of the aptamers expressed as monomers for their cognate bait are slightly higher than the apparent affinities of the dimers for either bait alone. This difference in affinity cannot be accounted for by a decreased expression level of the dimers as compared to that of the monomeric aptamers (western blot experiments, not shown).

ii) Test for Forced Interaction:

In order to establish whether the dimers have the capacity to force interaction between Cdk2 and Bax, use was made of an elaborated version of the two-hybrid assay in which a third protein is expressed in addition to a bait and a prey (FIG. 12). In these experiments, four dimers were cloned into pBC104, a yeast vector that directs the inducible expression of proteins fused to a nuclear localization sequence. Cdk2 (and other control proteins) were expressed as baits (fused to LexA) and Bax as a prey (fused to a nuclear localization sequence and an activation domain) The forced interaction activities were examined in an interaction mating assay (FIG. 10). All four dimers clearly elicited an interaction phenotype when co-expressed with LexA-Cdk2 and Act-Bax. These two proteins did not exhibit an interaction phenotype in presence of the two monomeric aptamers. None of the four dimers induced an interaction phenotype when an unrelated bait (LexA-Max) was expressed with Act-Bax.

iii) Further Experiments

The forced interaction between Cdk2 and Bax can be confirmed by other methods. Immunoprecipitation experiments are performed from the yeast strains used in FIG. 10. Whenever the yeast show an interaction phenotype, co-precipitation of the prey with the bait (or vice-versa) should be possible.

Furthermore, with the hypothesis that Cdk2 may exert its kinase activity on Bax (that arbors putative Cdk2 phosphorylation sites), western blot experiments can also be performed to examine putative slower migrating forms of Bax that could be reminiscent of a phosphorylation.

Materials and Methods

This section details the materials and methods used in the preceding examples.

Identification of a Higher Affinity Variant

The V region of anti-Cdk2 aptamer 10 was amplified from the original library vector (1) following a mutagenic PCR protocol as described (11). The purified amplified products were ligated into the RsrII-cut library vector, pJM-1 (1), which directs their conditional expression under the control of the Gal1 promoter, and introduced the ligation mix into E. coli DH5α. Plasmid DNA was prepared from a pool of 15,000 independent colonies. This pool was transformed (12) into EGY48 that contained LexA-Cdk2 (1) and pRB1840 (13) to obtain 40,000 transformants on Ura⁻His⁻Trp⁻ glucose plates. These transformants were replica plated onto Ura⁻His⁻Trp⁻ glucose/Xgal and Ura⁻His⁻Trp⁻ galactose/Xgal plates. After two days at 30° C., the 16 colonies that showed a blue color were transferred onto Ura⁻His⁻Trp⁻ glucose plates. These master plates were replica plated onto Ura⁻His⁻Trp⁻ glucose/Xgal and Ura⁻His⁻Trp⁼³¹ galactose/Xgal plates, and confirmed that 12 of the initial 16 colonies again displayed galactose-dependent blue color. The aptamer-encoding plasmids were rescued from these strains (14) and the plasmids were reintroduced into EGY48. 7 out of these 12 plasmids conferred an interaction phenotype on galactose-but not on glucose-containing medium.

Construction of Fusion Proteins

Modifiers. DNA encoding the hect domain of yeast RSP5 by PCR was isolated using the oligonucleotides 5′-ATATCTCGAGATTAAAGTACGTAGAAAGAAC-3′ and 5′-ATATGTCGACGGATCCTCATTCTTGACCAAACCCTATG-3′which respectively contained an XhoI site, and BamHI and SalI sites. The PCR product was subcloned into XhoI-cut pJG4-4 (ref. 42), which contains a Trp1 marker, a 2 μ replication origin, and that directed the expression of native proteins under the control of the GAL1 promoter, to create pJG4-4 (hect). TrxA and peptide aptamers were amplified using the oligonucleotides:

5′-GGAGGCGAATTCGCCGCCACCCATGGCCGATAAAATTATTCACCTGACTGACG-3′
and
5′-ATATCTCGAGCGCCAGGTTAGCGTCGAGGAAC-3′,

which contained respectively an EcoRI site followed by an initiator codon in a Kozak context, and an XhoI site. The PCR products were inserted into EcoRI/XhoI-cut pJG4-4 (hect). To express the hect domain only, the 5′ oligonucleotide:

5′-ATATGAATTCGCCGCCACCATGGCCATTAAAGTACGTAGAAAGAACATTTTTGAG-3′,

was used, which contained an EcoRI site and an initiator codon in a Kozak context with the above-described 3′ oligonucleotide to PCR the hect domain from RSP5. This fragment was introduced into EcoRI/XhoI-cut pJG4-4. The mutant hect domain was constructed using the Transformer kit from Clontech, according to the manufacturer's instructions, using the mutagenic oligonucleotide:

5′-GCCAAAATCTCACACAGCTTTTAACAGAGTTG-3′

to change the hect active site cysteine to alanine, and the selection oligonucleotide:

5′-CGCTAACCTGGCGCCTAGGATTAAAGTACGTAG-3′

to change to the XhoI site on the vector to an AvrII site.

For experiments with Myc-tagged ubiquitin, the modifiers were expressed from another vector. To this end, we amplified 5-hect, 8-hect, and 10M-hect fusions described above using oligonucleotides:

5′-ATATGTCGACGGATCCTCATTCTTGACCAAACCCTATG-3′
and
5′-GGAGGCGAATTCGCCGCCACCCATGGCCGATAAAATTATTCACCTGACTGACG-3′.

The amplified products were introduced into EcoRI/XhoI cut pBC103, a plasmid that carries a Ura3 marker, a 2 μ replication origin, and the GAL1 promoter.

Myc-ubiquitin. Yep105 contains a Trp1 marker and a 2 μ replication origin and directs the expression of a Myc-tagged synthetic yeast ubiquitin gene under the control of the copper-inducible CUP1 promoter (15).

LexA-7Lys-Cdk2. To start, the bait plasmid encoding LexA-Cdk2 (1) was used. DNA that encoded a stretch of 7 lysines was constructed by annealing the oligonucleotides:

5′-AATTGAAGAAGAAAAAAAAGAAAAAGC-3′
and
5′-AATTGCTTTTTCTTTTTTTTCTTCTTC-3′

and this duplex was introduced into the EcoRI site of the bait plasmid. The ligation mixture was treated with EcoRI, it was introduced into E. coli XL-1 blue, and cells that bore plasmids containing the insert were identified by PCR.

Transporters. TrxA and anti-Cdk2 peptide aptamers were amplified as described in “modifiers”. The PCR products were introduced into EcoRI/XhoI cut pBC103 (mentioned above) and pBC104, which also carried a Ura3 marker, a 2 μ replication origin, a Gal1 promoter, and that directed the synthesis of NLS-aptamers respectively. pJM-C6, -C7, -N2 and -N3, the plasmids that direct the synthesis of non-NLS- and NLS-tagged anti-Ste5 aptamers, are described elsewhere (7).

Yeast Manipulations

Interaction mating assays were performed as described (16) using the EGY48 strain for the preys and the EGY42 strain for the baits and reporter plasmids (17). Plasmids pSH18-34, pJK103, and pRB1840 (carrying respectively 8, 2, and 1 LexA operators upstream of a Gal1-lacZ fusion gene) were used in FIG. 1a, the pSH18-34-derived LexAop-GFP reporter (Display Systems Biotech, Copenhagen) in FIG. 1c and pSH18-34, in FIG. 2.

To measure interaction phenotypes with the LexAop-GFP reporter gene, overnight liquid cultures were grown from diploid exconjugants in Ura-His-Trp-galactose liquid medium. Fluorescence was measured with a FACStar plus (Becton-Dickinson) illuminated with two argon lasers tuned to 488 nm and to multiline UV. Recordings were made with a 530−/+15 nm filter to measure yeast fluorescence. The FL3-2 voltage (background) was set using yeast that did not show an interaction phenotype. 30,000 cells were analyzed for each interaction, and mean fluorescence of the yeast population above background was determined using the CellQuest software package (Becton-Dickinson).

To perform the modifier assays the EGY48 yeast strain was transformed with different combinations of targets and aptamer-hect fusions (12). Transformants were plated onto His⁻Trp⁻ glucose plates, then colonies were grown overnight in 4 ml of His⁻Trp⁻ galactose medium. For experiments with Myc-ubiquitin, EGY48 was transformed with aptamer-hect fusions and Yep105, and EGY42 was transformed with LexA-Cdk2 and pSH18-34. The strains obtained were mated and diploid exconjugants were selected on Ura-His-Trp-Leu-glucose medium. Liquid cultures were inoculated into Ura-His-Trp-Leu-galactose liquid medium, in which 100 μM CuSO4 was added or not.

For western analysis (below), equal amounts of yeast were pelleted in logarithmic growth phase and the pelleted yeast was treated with zymolase (Seikagaku Ltd.) at 1 mg/ml in 50□1 of (1M sorbitol, 0.5M Sodium citrate, 0.5 m EDTA, 1M DTT, 1M Potassium phosphate, 0.1M PMSF) for 1 hr at 30° C. and lysed with SDS PAGE sample buffer.

Immunoassays

Western blots were performed following SDS-PAGE as described (18) with rabbit anti-LexA serum (19) or rabbit anti-TrxA serum (20) secondary antibodies coupled to alkaline phosphatase, and NBT and BCIP as substrates. The blots were scanned with an optical scanner. For the Myc-Ubiquitin experiments, an ECF substrate (Amersham Pharmacia Biotech) was used and the blot was scanned with a phosphoimager (Molecular Dynamics). For immunofluorescence, aptamer expression was induced by growth in galactose for 3 hr and the cells were fixed by adding formaldehyde (3.7% final concentration) to the medium for 90 min. Samples were probed with a polyclonal rabbit anti-LexA antibody (UBI, New York), followed by affinity purified Texas Red- or fluorescein-conjugated secondary antibodies (Jackson ImmunoResearch Laboratories, PA), essentially as described (20).

Construction of Aptamer Dimers:

All PCR reactions described below were performed with a high-fidelity Vent polymerase, using as templates the aptamer library plasmids pJM-1 containing the coding sequences of the Cdk2 aptamer 10M or the Bax aptamer B16m103 (also referred to herein as M103)

Selection of the high affinity Bax aptamer B16M103: Selection of the parental Bax aptamer B16 is described in Example 4. Aptamer B16 comprises a 20 amino-acid variable region displayed in a thioredoxin platform as previously described (ref.1). The variable region of B16 has the amino acid sequence:

| | |
|---|---|
| Variable region B16: | PRGAPMWMRWVCQMLETMFL |

Mutagenic PCR was carried out on the variable region of B16 as described for Cdk2 aptamers. Amongst the obtained mutants, aptamer B16M103 was selected on the basis of high affinity and high selectivity for Bax (FIGS. 5, 6 and 7). The anti-Bax aptamer B16M103 (also designated “M103”) comprises a variable region having the amino acid sequence shown below, embedded in a thioredoxin platform as previously described (ref. 1):

| | |
|---|---|
| Variable region M103: | PRGAPMWLRCVCQMLETKFL |

Preparation of Dimers without linker: DNA encoding the aptamer destined to position 1 was PCR-amplified using the oligonucleotides olHAaptsens and olTrxend1. The PCR product was digested with EcoRI and NheI. DNA encoding the aptamer destined to position 2 was PCR-amplified using the oligonucleotides olNolink and olTrxend2. The PCR product was digested with NheI and XhoI. The prey vector pJG4-5 (17) was digested with EcoRI and XhoI and mixed with both digested PCR products. One step ligations were performed (See FIG. 15)

Dimers with a HA linker: DNA encoding the aptamer destined to position 1 was PCR-amplified using the oligonucleotides olHAaptsens and olTrxend1. The PCR product was digested with

EcoRI and NheI. DNA encoding the aptamer destined to position 2 was PCR-amplified using the oligonucleotides olLinkHA and olTrxend2. The PCR product was digested with NheI and XhoI. The ligation of both digested PCR products into pJG4-5 was performed as described above (See FIG. 14).

Dimers with a 5 Gly or a (4Gly−1Ser)×3 linker: DNA encoding the aptamer destined to position 1 was obtained as described above. DNA encoding the aptamer destined to position 2 was PCR amplified using the oligonucleotides olApt2 and olTrxend2. The PCR product was digested with NcoI and XhoI. DNA encoding the 5 Gly linker was obtained by hybridizing the oligonucleotides olPoly5Gly and olPoly5Glyrev. DNA encoding the (4Gly−1Ser)×3 linker was obtained by hybridizing the oligonucleotides olGly4Ser and olGly4Serrev. The resulting duplexes contained a NheI cohesive end on their 5′ terminus and a NcoI cohesive end on their 3′ terminus. One-step ligations of EcoRI/XhoI-cut pJG4-5, both digested PCR products, and either one of the duplexes encoding the linkers were performed (See FIG. 13)

Oligonucleotide sequences:

| | |
|---|---|
| OlHAaptsens: | 5′ CGGGGTACCTTTGGGTCCTACCCTTATGATGTG 3′ |
| olTrxEnd1: | 5′ ATTTAAGCTAGCGGCCAGGTTAGCGTCGAGGAAC 3′ |
| olNoLink: | 5′ TTAATAGCTAGCATGAGCGATAAAATTATTCACC 3′ |
| olTrxEnd2: | 5′ AATATCTCGAGCTAGGCCAGCTTAGCGTCGAGG 3′ |
| olLinkHA: | 5′ TTAATAGCTAGCTTTGGGTCCTACCCTTATGATGTG 3′ |
| olApta2: | 5′ TTATTCCATGGTATGAGCGATAAAATTATTCACC 3′ |
| olPoly5Gly: | 5′ CTAGCGGTGGTGGTGGCGGC 3′ |
| olPoly5Glyrev: | 5′ CATGGCCGCCACCACCACCG 3′ |
| olGly4Ser: | 5′ CTACCGGTGGTGGTGGCTCCGGTGGTGCTGGCTCCGGTGCTGGTGGCTCC 3′ |
| olGly4Serrev: | 5′ CATGGGAGCCACCACCACCGGAGCCACCACCACCGGAGCCACCACCACCG |

Interaction Mating Assays for Aptamer Dimers:

i) Binding assay: The experimental procedure for the interaction mating assays shown in FIGS. 8 and 9 is described above in “yeast manipulation”. In all experiments containing dimers (FIGS. 8, 9 10), the reporter plasmid used is pSH18-34T. This plasmid is pSH18-34, with an ADH terminator placed behing the lacZ gene to stabilize the lacZ messenger. Hence pSH18-34T is a more sensitive reporter plasmid than pSH18-34.

ii) Forced Interaction assays: All the aptamer dimer plasmids described above (except those containing the HA linker) were digested with EcoRI and XhoI and ligated into EcoRI/XhoI-cut pBC104. The two dimers containing the HA linker were built in pBC104 in the same way they were built in pJG4-5 (see above). DNA encoding the 10M and B16 m103 aptamers was PCR-amplified as described in “modifiers”. The PCR products were digested with EcoRI and XhoI and ligated into EcoRI/XhoI-cut pBC104.

The EGY42 strain was transformed with the bait plasmids and the pSH18-34T reporter plasmid. The strain was transformed with the Bax prey plasmid and the different pBC104 constructs. Interaction mating assays were performed as described (16).

### REFERENCES

1. Colas, P., Cohen, B., Jessen, T., Grishina, I., McCoy, J. & Brent, R. (1996) Nature 380, 548-550.

2. Xu, C. W., Mendelsohn, A. & Brent, R. (1997) Proc. Natl. Acad. Sci. USA 94, 12473-12478.

3. Cohen, B. A., Colas, P. & Brent, R. (1998) Proc. Natl. Acad. Sci. USA 95, 14272-14277.

4. Fabrizzio, E., Le Cam, J., Polanowska, J., Kakzorek, M., Lamb, N., Brent, R. & Sardet, C. (1999) Oncogene 18, 4357-4363.

5. Butz, K., Denk, C., Ullmann, A., Scheffner, M., & Hoppe-Seyler, F. Proc. Natl. Acad. Sci. USA 97, 6693-6697

6. Kolonin, M. G. & Finley, R. L., Jr. (1998) Proc. Natl. Acad. Sci. USA 95, 14266-14271.

7. Geyer, C. R., Colman-Lerner, A. & Brent, R. (1999) Proc. Natl. Acad. Sci. USA 96, 8567-8572.

8. Norman, T. C., Smith, D. L., Sorger, P. K., Drees, B. L., O'Rourke, S. M., Hughes, T. R., Roberts, C. J., Friend, S. H., Fields, S. & Murray, A. W. (1999) Science 285, 591-595.

9. Blum, J. H., Dove, S. L., Hochschild, A., Mekalanos, J. J. (2000) Proc. Natl. Acad. Sci USA 97, 2241-2246.

10. Colas, P. (2000) Curr. Opin. Chem. Biol. 4, 54-59.

11. Cadwell, R. C. & Joyce, G. F. (1992) PCR Methods Appl. 2, 28-33.

12. Gietz, D., St. Jean, A., Woods, R. A. & Schiestl, R. H. (1992) Nuc. Acids Res. 20, 1425.

13. Estojak, J., Brent, R. & Golemis, E. A. (1995) Mol. Cell. Biol. 15, 5820-5829.

14. Finley, R. L., Jr. & Brent, R. (1995) in DNA Cloning, Expression Systems: A Practical Approach, eds. Hames, B. D. & Glover, D. M. (Oxford University Press, Oxford), pp.169-203.

15. Ellison, M. J. & Hochstrasser, M. (1991) J. Biol. Chem. 266, 21150-21157.

16. Finley, R. L., Jr. & Brent, R. (1994) Proc. Natl. Acad. Sci. USA 91, 2980-12984.

17. Golemis, E. A. & Brent, R. (1992) Mol. Cell. Biol. 12, 3006-3014.

18. Ausubel, F. M., Brent, R., Kingston, R. E., Moore, D., Seidman, J. G. & Struhl, K. (1987-1997) (Greene and Wiley-interscience, New York).

19. Brent, R. & Ptashne, M. (1984) Nature 312, 612-615.

20. LaVallie, E. R., DiBlasio, E. A., Kovacic, S., Grant, K. L., Schendel, P. F. & McCoy, J. M. (1993) Biotechnology (NY) 11, 187-193.

21. Ferrigno, P. & Silver, P. A. (1999) Methods Cell. Biol. 58, 107-122.

22. Scheffner, M., Nuber, U. & Huibregtse, J. M. (1995) Nature 373, 81-83.

23. Huibregtse, J. M., Scheffner, M., Beaudenon, S. & Howley, P. M. (1995) Proc. Natl. Acad. Sci. USA 92, 2563-2567.

24. Jennissen, H. P. (1995) Eur. J. Biochem. 231, 1-30.

25. Katti, S. K., LeMaster, D. M. & Eklund, H. (1990) J. Mol. Biol. 212, 167-184.

26. Silver, P. A., Brent, R. & Ptashne, M. (1986) Mol. Cell. Biol. 6, 4763-4766.

27. Mahanty, S. K., Wang, Y., Farley, F. W., Elion, E. A. (1999) Cell 98, 501-512.

28. Brent, R. & Ptashne, M. (1985) Cell 43, 729-736.

29. Oertel-Buchheit, P., Lamerichs, R. M., Schnarr, M. & Granger-Schnarr, M. (1990) Mol. Gen. Genet. 1, 40-48.

30. Wang, G., Yang, J. & Huibregtse, J. M. (1999) Mol. Cell. Biol. 19, 342-352.

31. Gosink, M. M. & Vierstra, R. D. (1995) Proc. Natl. Acad. Sci. USA 92, 9117-9121.

32. Hershko, A. (1997) Curr. Opin. Cell. Biol. 9, 788-799.

33. Mahaffey, D. T., Yoo, Y. & Rechsteiner, M. (1995) FEBS Lett. 370, 109-112.

34. Terrell, J., Shih, S., Dunn, R. & Hicke, L. (1998) Mol. Cell 1, 193-202.

35. Pelham, H. R. (1996) Cell. Struct. Funct. 21, 413-419.

36. Schneider, S., Buchert, M., Georgiev, O., Catimel, B., Halford, M., Stacker, S. A., Baechi, T., Moelling, K. & Hovens, C. M. (1999) Nature Biotech. 17, 170-175.

37. Haucke, V. & Lithgow, T. (1997) J. Bioenerg. Biomembr. 29,11-17.

38. Boutin, J. A. (1997) Cell Signal. 9, 15-35.

39. Vita, C. (1997) Curr. Opin. Biotechnol. 8, 429-434.

40. Gilbert, W., de Souza, S. J. & Long, M. (1997) Proc. Natl. Acad. Sci. USA 94, 7698-7703.

41. Beerli, R. R., Wels, W., Hynes, N. E., (1994), J. Biol. Chem., 269, 39, 23931-23936

42. Gyuris, J., Golemis, E., Chertkov, H & Brent, R., (1993), Cell 75, 791-803

43. Eklund et al., EMBO J. 3: 1443-1449 (1984)

44. Holmgren, J., Biol. Chem., 264: 13963-13966 (1989)

1. Process for specifically modulating the properties of an intracellular target molecule T, and/or of a cellular component C which interacts directly or indirectly in a cell with T, said process comprising:
introducing into a cell a chimeric molecule, a so-called <<targeted effector>>, comprising:
a recognition moiety R, comprising a peptide V having a length of five to sixty amino acids, preferably ten to forty amino acids, conformationally constrained by covalent linking to a platform, said recognition moiety R having the capacity to specifically interact, within the cell, with a site on an intracellular target molecule T, the interaction with T occurring with an affinity A₁, and
an effector moiety, E covalently linked to said recognition moiety R, E being a molecule, or a portion thereof, which has an initial capacity to exert an effect on at least one molecule M, and which when it is covalently linked to R, acquires the capacity to exert the effect on the intracellular target molecule T,
wherein the targeted effector interacts with T with a second affinity A₂, the affinity A₁ or the affinity A₂ corresponding to a K_{d} of less than 1×10⁻⁸ M, and the properties of T and/or of C are specifically modulated by the effector moiety E.

2. Process according to claim 1 wherein the peptide V has a length of six to forty amino acids,

3. Process according to claim 1 wherein the peptide V comprises a random peptide.

4. Process according to any one of claims 1 to 3 wherein the platform comprises or consists of a protein.

5. Process according to any one of claims 1 to 4 wherein the platform is heterologous with respect to the peptide V.

6. Process according to claim 5 wherein the peptide V is embedded in the platform.

7. Process according to claim 5 wherein said platform is thioredoxin (TRX) or a TRX-like protein.

8. Process according to claim 1 wherein the binding affinity A₁ corresponds to a K_{d} comprised between 1×10⁻⁹ M and 1×10⁻¹² M.

9. Process according to claim 1 wherein R is a mutant of a parent recognition moiety Rₐ, said parent recognition moiety Rₐ having the capacity to specifically interact with a site on said intracellular target molecule T, the interaction between Rₐ and T occurring with an affinity A₃, wherein the K_{d} corresponding to A₃ is greater than the K_{d} corresponding to A₁.

10. Process according to claim 1 wherein E has the initial capacity to exert an effect in cis and/or in trans on M, and, when it is covalently bound to R, acquires the capacity to exert the effect in trans on T.

11. Process according to claim 1 wherein the effector moiety E comprises an enzyme, a co-factor, an addressing signal, a transcription regulatory protein, a tracer protein, a molecule having therapeutic or diagnostic properties, a second recognition moiety, a second targeted effector, a radionuclide, a chemical modifier.

12. Process according to claim 1 wherein the effector moiety E comprises a second recognition moiety R₂ having the capacity to specifically interact, within a cell, with a site on an intracellular target molecule T₂, T₂ being a target molecule which is distinct from T

13. Process according to claim 1 wherein the effector moiety E is not a transcription activator.

14. Process according to claim 6 wherein the effector moiety E is covalently bound to the amino or carboxy terminal of the recognition moiety R.

15. Process according to claim 1 wherein the identity of T and/or C is predetermined.

16. Process according to claim 1 or 15, wherein the target molecule T or the cellular component C comprises a protein, a nucleic acid, a carbohydrate, a phosphorylated molecule, a lipid, or a combination of any of the foregoing.

17. Process according to claim 1 wherein the target protein T and/or the cellular component C is/are endogenous to the cell.

18. Process according to claim 1 wherein the target protein T is a protein comprising at least two functionally distinct protein domains, Tₐ and T_{b}, and wherein the recognition moiety R specifically binds to domain Tₐ and the effector moiety E exerts the effect on domain T_{b}.

19. Process according to claim 1 wherein R has the capacity to specifically interact, within the cell, with a site on a predetermined range of target molecules, including T, and the effector moiety E, when it is covalently bound to R, acquires the capacity to exert the effect in trans in the cell on the said predetermined range of intracellular target molecules.

20. Process according to claim 1 wherein R interacts with a site occurring within the cell exclusively on T.

21. Process according to claim 1 wherein the recognition moiety R, in the absence of the effector moiety E, is neutral with respect to the function of the target protein T.

22. Process according to claim 1 wherein the property of T and/or C which is modulated by E is a chemical, biochemical, physical and/or functional property.

23. Process according to claim 22 wherein the modulation of the chemical, biochemical, physical, or functional properties of the target protein T or the cellular component C gives rise to a phenotypical change in the cell or organism, said phenotypical change being optionally selectable or detectable.

24. Process according to claim 22 wherein the change in the chemical properties of T and/or C comprises the formation or breakage of covalent or non-covalent bonds.

25. Process according to claim 22 wherein the change in the physical properties of T and/or C comprises a change in conformation, or in sub-cellular localisation.

26. Process according to claim 22 wherein the change in the functional properties of T and/or C comprises the enhancement or reduction in the efficiency of one or more of the functions of T and/or C, or the abolition of a function, or the creation of a new function.

27. Process according to claim 22 wherein the modulation gives rise to one or more of the following effects:
homo- or heterodimerisation of T or C
inhibition of homo- or heterodimerisation of T or C
destruction of T or C;
stabilisation of T or C;
labeling of T or C;
ability of T or C to interact with a new partner;
localisation of T or C to a subcellular compartment to which T or C are not otherwise transported in the cell;
localisation of T or C to a subcellular compartment to which T or C are otherwise transported to a lesser degree in the cell;
exposure of T or C at the cell membrane;
internalisation of T or C;
provoking the association of T or C with natural intracellular partners;
prevention of the association of T or C with natural intracellular partners;
cleavage of T or C;
secretion of T or C;
modification of cell cycle dynamics.

28. Process according to claim 1 wherein the cell is a eukaryotic cell.

29. Process according to claim 1 wherein the cell is a prokaryotic cell.

30. Process according to claim 1 wherein the targeted effector is introduced into the cell by expression of a DNA sequence encoding said targeted effector as a fusion protein.

31. Process according to claim 1 wherein the targeted effector is introduced into the cell in purified form using a cell permeable agent such as a protein transduction domain (P.T.D).

32. Process according to claim 1 wherein, in a given set of conditions in the cell, the effector moiety E has the capacity to exert an effect on T when it is covalently bound to the recognition moiety, but cannot exert the said effect on T or C when it is not covalently bound to the recognition moiety, in said conditions.

33. Process according to claim 32 wherein the inability of the effector moiety E to exert the effect on T or C in the cell when it is not covalently bound to the recognition moiety is due to the inherent specificity of E or T

34. Process according to claim 33 wherein the effector moiety E is an enzyme whose inherent specificity prevents the target protein T from acting as substrate for E when E is not covalently linked to the recognition moiety.

35. Process according to claim 32 wherein the inability of the eeffector moiety E to exert said effect on T or C in said cell when it is not covalently bound to the recognition moiety is due to lack of co-expression of E and T and/or C.

36. Process according to claim 32 wherein T and/or E exhibit a temporal or spatial specificity which prevents the effector moiety E and the target molecule T from associating with each other when E is not covalently linked to the recognition moiety R.

37. Process according to claim 1 wherein the effector moiety E initially has the capacity to direct an intracellular molecule M in cis to a predetermined sub-cellular compartment, and acquires the capacity to direct the target molecule T in trans to the said sub-cellular compartment when E is covalently linked to R.

38. Process for the production of a targeted effector having the capacity to specifically modulate the properties of an intracellular target molecule T, and/or a cellular component C which interacts directly or indirectly in a cell with T, said process comprising the steps:
a) production of a random pool of peptides, so-called recognition moieties R,
b) screening of the random pool produced in (a) against T in a cell, in conditions suitable to allow identification of recognition moieties R capable of interacting with T,
c) optionally contacting the moieties selected in (b) with proteins other than T to determine the specificity range of each of said moieties, and to identify moieties having a desired specificity range,
d) covalent linkage of the recognition moieties R to an effector moiety E, E being a molecule which initially has the capacity to exert a predetermined effect on at least one intracellular component M, step (d) being carried out either prior to step (b), or after step (e),
e) verification of the affinity A, with which the recognition moiety R interacts with T, or of the affinity A₂ with which the targeted effector, interacts with T,
f) if both of A₁ and A₂ correspond to K_{d} values greater than 1×10⁻⁸ M, alteration of the binding region of the recognitio moiety to adjust the binding affinity of the interaction between T and the selected moiety so that the K_{d} becomes less than 1×10⁻⁸ M.

39. Process according to claim 38 wherein step (b) comprises the screening of the pool of random peptides in a cell for a detectable or selectable phenotypic change occurring via an interaction with an unknown endogenous target T, or via a modification of T by E when step (d) is carried out prior to step (b).

40. Process according to claim 39 further comprising the step of determining the identity of T by screening selected recognition moieties against molecules known to have the capacity to give rise to said phenotypic change.

41. Process according to claim 39 further comprising the step of determining the identity of T by performing a two-hybrid screen using R or the targeted effector R-E as bait.

42. Process according to claim 38 wherein step (b) comprises the screening of the pool of random peptides in a cell against a known target molecule T, interaction with T giving rise to a detectable or selectable change.

43. Process according to claim 38 wherein step (b) comprises detection of reporter gene transcription in a two-hybrid interaction trap system, candidate target proteins being co-expressed in the cell with the recognition moieties.

44. Process for conferring on an effector moiety E the ability to specifically modulate the properies of an intracellular protein T, or an intracellular component C which interacts directly or indirectly with T,
said process comprising the steps of
i) covalently linking the effector moiety E to a recognition moiety R,
wherein R comprises a molecule having the capacity to specifically interact, within a cell, with a site on an intracellular target molecule T, the interaction with T occurring with an affinity A₁ which corresponds to a K_{d} value of less than 1×10⁻⁸ M, and
E being a molecule which has an initial capacity to exert an effect on at least one molecule M, and which when it is covalently linked to R, acquires the capacity to exert the effect on the intracellular target molecule T,
ii) optionally optimising the affinity of the interaction between T and R by altering the chemical composition of the binding region of R to provide an affinity in the desired range.

45. Chimeric molecule, so-called <<targeted effector>>, said targeted effector comprising
a proteinaceous recognition moiety R, comprising a peptide V having a length of five to sixty amino acids, preferably ten to forty amino acids, conformationally constrained by covalent linking to a platform, said recognition moiety R having the capacity to specifically interact, within a cell, with a site on an intracellular target molecule T, the interaction with T occurring with an affinity A₁, and
an effector moiety, E, covalently linked to R, E being a molecule which has an initial capacity to exert an effect on at least one molecule M, and which when it is covalently linked to R, acquires the capacity to exert the effect on the intracellular target molecule T, wherein the targeted effector interacts with T with an affinity A₂, the affinity A, or the affinity A₂ corresponding to a K_{d} value of less than 1×10⁻⁸ M.

46. Chimeric molecule according to claim 45 wherein the peptide V comprises a random peptide.

47. Chimeric molecule according to claim 45 or 46 wherein the platform comprises or consists of a protein.

48. Chimeric molecule according to claim 47 wherein the platform is heterologous with respect to the peptide V.

49. Chimeric molecule according to claim 48 wherein said platform is thioredoxin (TRX) or a TRX-like protein.

50. Chimeric molecule according to claim 45 wherein the binding affinity A₁ corresponds to a K_{d} value of less than 1×10⁻⁹ M.

51. Chimeric molecule according to any one of claims 45 to 50 wherein the effector moiety E comprises an enzyme, a co-factor, an addressing signal, a transcription regulatory protein, a tracer protein, a target for intracellular enzymes, a molecule having therapeutic or diagnostic properties, a second recognition moiety, a second targeted effector, a radionuclide, a chemical modifier.

52. Chimeric molecule according to claim 51 wherein the effector moiety E comprises a second recognition moiety R₂ having the capacity to specifically interact, within a cell, with a site on an intracellular target molecule T₂, T₂ being a target molecule which is different from T

53. Nucleic acid encoding a chimeric protein according to claim 45 operably linked to regulatory sequences for expression in a eukaryotic cell.

54. Vector capable of stably introducing a nucleic acid according to claim 53 into a prokaryotic or eukaryotic cell.

55. Cell containing a chimeric molecule according to claim 45, or a nucleic acid according to claim 53.

56. Cell according to claim 55 which is a eukaryotic cell.

57. Cell according to claim 55 which is a prokaryotic cell.

58. Pharmaceutical composition comprising a chimeric molecule according to claim 45, or a nucleic acid according to claim 53, in association with a pharmaceutically acceptable excipient.

59. Chimeric molecule according to claim 45 for use in therapy.

60. Nucleic acid according to claim 53 for use in therapy.

61. Use of a chimeric protein according to claim 45 or a nucleic acid according to claim 53 for the preparation of a medicament for the treatment of:
microbial infections, including viral infections, and fungal infections;
immunological disorders;
neurological disorders;
metabolic disorders;
psychiatric disorders;
myopathies;
genetic disorders;
cancer;
cardiovascular disorders;
dental disorders;
aesthetic disorders.

62. Use of a chimeric protein according to claims 45 to 52, or a nucleic acid according to claim 53 for specifically modulating the properties of an intracellular target molecule T, and/or a cellular component C which interacts directly or indirectly in a cell with T.

63. Intracellular recognition molecule R, comprising a proteinaceous recognition domain, conformationally constrained by covalent bonding to a platform,
said recognition molecule R having the capacity to specifically interact, within a cell, with a site on a predetermined intracellular target molecule T, the interaction with T occurring with an affinity corresponding to a K_{d} value of less than or equal to 5×10⁻⁹ M.

64. Intracellular recognition molecule R according to claim 63 wherein the recognition domain comprises or consists of a peptide having a length of five to sixty amino acids, preferably ten to forty amino acids.

65. Intracellular recognition molecule R according to claim 64 wherein the peptide recognition domain comprises a random peptide.

66. Intracellular recognition molecule R according to any one of claims 63 to 65 wherein the platform comprises or consists of a protein.

67. Intracellular recognition molecule R according to claim 64 or 66, wherein the platform is heterologous with respect to the recognition domain.

68. Intracellular recognition molecule R according to claim 67 wherein said platform is thioredoxin (TRX) or a TRX-like protein.

69. Intracellular recognition molecule according to any one of claims 63 to 69, wherein the affinity of the interaction with T corresponds to a K_{d} value comprised between 1×10⁻⁹ M and ×10⁻¹⁴ M.

70. Intracellular recognition molecule according to any one of claims 63 to 69 wherein the intracellular target molecule T with which R has the capacity to specifically interact is chosen from a cyclin-dependent kinase, a pro-apoptotic protein.

71. Intracellular recognition molecule according to claim 70 wherein the intracellular target molecule T is Cdk2.

72. Intracellular recognition molecule according to claim 71 wherein the peptide recognition domain comprises or consists of a mutant of the amino acid sequence QVWSLWALGWRWLRRYGWNM, said mutant having from one to five, preferably one to three amino acid changes with respect to said sequence.

73. Intracellular recognition molecule according to claim 72 wherein the peptide recognition domain comprises or consists of the amino acid sequence QVWSSWALGWRWLRRYGWGM.

74. Intracellular recognition molecule according to claim 70 wherein the intracellular target molecule T is Bax.

75. Intracellular recognition molecule according to claim 74 wherein the peptide recognition domain comprises or consists of a mutant of the amino acid sequence PRGAPMWMRWVCQMLETMFL, said mutant having from one to five, preferably one to three amino acid changes with respect to said sequence.

76. Intracellular recognition molecule according to claim 72 wherein the peptide recognition domain comprises or consists of the amino acid sequence PRGAPMWLRCVCQMLETKFL.

77. Oligomeric intracellular recognition molecule, comprising from two to four intracellular recognition molecules R according to claim 63, said recognition molecules being covalently linked to each other, either directly or via a linker.

78. Oligomeric intracellular recognition molecule according to claim 77 comprising two intracellular recognition molecules R.

79. Dimeric intracellular recognition molecule, comprising two covalently linked intracellular recognition molecules R1 and R2,
wherein R1 and R2 each comprise a recognition domain V, V being a peptide having a length of five to sixty amino acids, conformationally constrained by covalent bonding to a platform,
R1 having the capacity to specifically interact, within a cell, with a site on a predetermined intracellular target molecule T1, and
R2 having the capacity to specifically interact, within a cell, with a site on a predetermined intracellular target molecule T2,
wherein R1 and R2 may be the same or different and T1 and T2 are distinct.

80. Dimeric intracellular recognition molecule according to claim 79, wherein R1 and R2 are identical, and the intracellular recognition molecule specifically and simultaneously binds to two identical but separate target molecules T1 and T2.

81. Dimeric intracellular recognition molecule according to claim 79, wherein R1 and R2 are different, and the intracellular recognition molecule specifically and simultaneously binds to two different and separate target molecules T1 and T2.

82. Dimeric intracellular recognition molecule according to claim 80 or 81, wherein the target molecules T1 and T2 do not detectably interact with each other in a cell in the absence of the recognition molecule.

83. Process for the identification of dimeric recognition moieties having the capacity to force an interaction between two target molecule T1 and T2, and/or a cellular component C which interacts directly or indirectly in a cell with T1 or T2 said process comprising the steps
a) production of a random pool of peptides, so-called recognition moieties R,
b) screening of the random pool produced in (a) against T1 and T2 in a cell, in conditions suitable to allow identification of recognition moieties R capable of interacting with T1 or T2,
c) optionally contacting the moieties selected in (b) with proteins other than T1 and T2 to determine the specificity range of each of said moieties, and to identify moieties having a desired specificity range,
f) covalent linkage of two thus-identified recognition moieties R1 and R2 to each other to provide a dimeric recognition moiety,
g) screening of the dimeric recognition moiety to identify moieties capable of interacting simultaneously with T1 and T2.
